(19) United States
(12) Patent Application Publication
(10) Pub. No.: US 2005/0197582 A1
(43) Pub. Date: Sep. 8, 2005

(54) Process and compositions for synthetic calibration of bio-photonic scanners

(76) Inventors: Scott Ferguson, East Spanish Fork, UT (US); Stephen J. Poole, Provo, UT (US); Douglas Stevenson, Santaquin, UT (US)

Correspondence Address: PATE PIERCE & BAIRD, 215 SOUTH STATE STREET, SUITE 550, PARKSIDE TOWER, SALT LAKE CITY, UT, 84111, US

(21) Appl. No.: 10/981,147

(22) Filed: Nov. 3, 2004

Related U.S. Application Data
(63) Continuation-in-part of application No. 10/946,367, filed on Sep. 20, 2004.
(60) Provisional application No. 60/546,112, filed on Feb. 19, 2004.
(60) Provisional application No. 60/545,806, filed on Feb. 19, 2004.

(51) Int. Cl. A61B 6/00

(52) U.S. Cl. 600/476

(57) ABSTRACT

A method, apparatus, and set of compositions are disclosed for calibrating a bio-photonic scanner. The scanner detects selected molecular structures of tissues, nondestructively, in vivo. The apparatus may include a computer, including processor and memory connecting to the scanner, including an illuminator to direct light nondestructively onto tissue in vivo, a detector to detect an intensity of a radiant response of the tissue to the light, and a probe to direct light onto the subject and receive a radiant response back into the detector. The apparatus is calibrated using a synthetic material to mimic the radiant response of live tissue, correcting for background fluorescence and elastic scattering. Dopants in a matrix of synthetic material mimic selected molecular structures of tissue. Matrix materials include a dilatant compound, and dopants include biological materials as well as K-type polarizing film powdered and mixed.

### 1. RELATED APPLICATIONS

This application is a continuation-in-part of U.S. patent application Ser. No. 10/946,367, filed on Sep. 20, 2004 for SYNTHETIC CALIBRATION STANDARD FOR PHOTONIC RESPONSE OF TISSUE, which claims the benefit of co-pending U.S. Provisional Patent Application Ser. No. 60/546,112, filed on Feb. 19, 2004 for SYNTHETIC CALIBRATION STANDARD FOR PHOTONIC RESPONSE OF TISSUES, and co-pending U.S. Provisional Patent Application Ser. No. 60/545,806, filed on Feb. 19, 2004 for BIO-PHOTONIC SCANNING CALIBRATION APPARATUS AND METHOD.

### BACKGROUND

2. The Field of the Invention

This invention relates to optical measurement of intensity of light and, more particularly, to novel systems and methods for calibrating detectors of Raman scattering.

3. The Background Art

Optical and electronic mechanisms have been developed to generate, detect, observe, track, characterize, process, manipulate, present, and otherwise manage characteristic signals representative of materials, properties, systems, and the like. In the world of engineering, many principles of physics operate predictably, repeatably, and in accordance with the plans and schemes of those harnessing those laws of physics and engineering. Accordingly, over time, the mathematics of analysis or prediction of the performance and behavior of physical systems has been developed to a fine art and a reliable science.

The application of mechanical and electronic apparatus, as well as optical systems, radiation (e.g. radar, light, etc.), and sound (e.g. ultrasonic scanning, sonar, etc.) have proven useful in monitoring many types of systems. Many systems that are tested or monitored, and other systems that are designed and controlled, rely upon the technologies that combine physical phenomena with mathematical representations of those phenomena and the processing power of computers. Add to this mix various systems for detecting physical behaviors, converting those behaviors into signals, and submitting those signals for processing to computers, and much of the technical world in which people operate can be designed, analyzed, constructed, observed, and otherwise rendered more understandable and useful.

In the biological sciences, instrumentation has proven extremely helpful in both diagnostics and treatments. Electrocardiograms, electroencephalographs, and the like record weak electromagnetic signals characterizing the operation of the heart, nervous system, and so forth. Similarly, ultrasonic images, x-rays, and the like provide insight and literal vision into certain biological processes. The CT scan, or computed tomography technology has likewise provided greatly enhanced abilities to image biological systems and processes.

Likewise, the field of chemistry has benefitted from technology including much instrumentation, including such devices as chromatographs, spectral analysis, and the like. In all this knowledge being gained and applied to the understanding and control of biological organisms, processes, and the like, a continuing need is the reliable calibration of instrumentation used for such tasks.

For example, systems for measurement of selected chemical compositions in biological tissue have been developed in recent years. Useful examples of such apparatus are disclosed in U.S. Pat. No. 5,873,831 issued Feb. 23, 1999 to Bernstein et al. and directed to a method and system for measurement of macular carotenoid levels, incorporated herein by reference. Likewise, a patent was issued for non-invasive measurement of other tissues as well. This work is documented in U.S. Pat. No. 6,205,354 B1 issued Mar. 20, 2001 to Gellermann et al. and directed to a method and apparatus for non-invasive measurement of carotenoids and related chemical substances in biological tissue, also incorporated herein by reference. Follow on work by substantially the same team of scientists resulted in a U.S. patent application Ser. No. 10/040,883 identified as Publication No. US2003/0130579A1 published Jul. 10, 2003, directed to a method and apparatus for Raman imaging of macular pigments, and incorporated herein by reference. This work or this entire body of work provides among other things for determination of levels of carotenoids of similar chemical compounds in tissues such as living skin. Certain methods and apparatus are disclosed for non-invasive, rapid, accurate, and safe determination of carotenoid levels. These determinations may be used as diagnostic information regarding cancer risk or as markers for conditions where carotenoids or other antioxidant compounds may provide diagnostic information. Thus, much of this work is directed to early diagnostic information and possible prevention or intervention.

In general, these processes rely on a technique of resonance Raman spectroscopy to measure levels of carotenoids in similar substances and tissue. In certain embodiments, a laser light is directed onto an area of tissue of interest. A small fraction of this scattered light is scattered inelastically by a process of Raman scattering in which energy is absorbed by selected molecules of interest, and is re-radiated at a different frequency from that of the incident laser light. The Raman signal may be collected, filtered, and measured. The resulting signal may then be analyzed in order to remove elastic scattering (e.g. reflectance) of the illuminating source light, as well as background fluorescence in order to highlight the characteristic peak identified as the Raman scattering signal.

In certain embodiments, a laser light source is passed into a probe system containing various lenses, beam splitters, and the like. Accordingly, coherent light from a laser source may be passed through this series of lenses and beam splitters to a mirrored surface through which the beam may pass on it way to impinging upon a subject (e.g. skin, macula, etc.) in order to generate a response. The responsive radiation passes back into the probe, is typically reflected off the beam splitter or partially silvered mirror to be redirected into a detector.

In one example, a spectrally selective system, such as a charge coupled device detects radiation (e.g. light waves, photons, etc.) according to intensity and frequency (reciprocally wavelength). Thus, the wavelengths and intensities may be processed in order to quantify the amount of irradiance occurring along a spectrum of frequencies or wavelengths.

The response to impinging, coherent light on tissues may thus be characterized by the amount of energy, the number photons, or the like arriving at a detector in response to a particular illumination source. One can imagine that such a device, if sufficiently precise might conceivably measure even down to an individual photon level of quantum variation in radiant energy response.

In order to implement such devices, a method and apparatus are needed that can reliably calibrate scanners (e.g. systems for illumination of subjects and retrieval of radiant responses thereto for processing) and for processors or computers to manipulate and otherwise process the data received therefrom. Several needs arise in attempting to project a laboratory device or laboratory curiosity into a medical and diagnostic field or into a marketplace for such instruments. For example, tissues vary by their nature and by the difference in organisms.

For example, tissues of plants may behave characteristically, and some particular average or normal value or range of values may be established for a particular variety of plant under certain conditions. Similarly, tissues of animals or people may be analyzed invasively or non-invasively in order to correlate certain characteristics thereof with the radiant responses of such tissues to illumination and Raman scattering. Averages are an interesting characteristic of a property of a population.

Nevertheless, the variation between electronic components is not negligible. Accordingly, any combination of electrical and optical components will have certain inherent characteristics. In operating a scanner, the electrical and electronic artifacts (e.g. errors, characteristics, anomalies, bias, and so forth) of the device in question need to be characterized in order to be factored out of measurements or calculations. Typically, the variations between any two devices produced need to be some how calibrated (e.g. measured, compensated, scaled, normalized, etc.) in order that an output by a particular device be repeatable between devices. That is, two or a hundred devices of a same design need to be able to produce the same or substantially the same value of a detected parameter when evaluating the same subject. That is, the skin of an individual scanned by two or a hundred different machines of the same design should provide substantially the same output value, within some reasonable repeatability (precision) and accuracy (reflection of true reality).

Thus, what is needed is an apparatus and method to calibrate individual scanners in order that the machine-to-machine variation can be factored out, resulting in an output from each machine that will be identical within some acceptable degree of variation, for a scan conducted on the same sample. Moreover, inasmuch as conditions change, such as temperature, humidity, chemistry, physical properties, and the like, over short times and long times in some expected, unexpected, predictable, or unpredictable manner, a machine needs to be calibrated to remove its own temporal (time wise) variations in operation.

That is, a scanning device operated on one day needs to be able to produce substantially the same output on another day or at some other time when exposed to the same identical condition in substantially the same subject. That is, the day-to-day variations or the time-to-time variability in outputs obtained from a particular device need to be calibrated out. That is, a method and apparatus are needed to calibrate a scanner in such a way as to factor out the vagaries of physics, chemistry, temperature, external conditions, and the like that may otherwise affect the output of a device. Thus, a method and apparatus for field calibration for a scanner would be an advance in the art.

To the extent possible, it would be an advance in the art to establish a process for processing signals received from a scanning device, in order that the hardware not be required to be adjusted. That is, for example, to the extent that various conditions can be monitored, or detected in a calibration process, then the output signals from such a device can simply be processed in order to correct the values of those signals, rather than actually correcting or altering any performance parameter, physical characteristic, or other control parameter associated with a scanning device. Thus, it would be an advance in the art to develop signal processing or computational processing of signal data obtained from a scanner in order to provide all the foregoing calibration benefits.

Biological materials are inherently highly variable. That is, a statistically significant sample over a properly identified population may have utility. Nevertheless, the portability of a sample may be problematic. For example, how does one normalize or calibrate two different machines on two different continents scanning two different populations in order that those devices read the same. Calibration samples taken from biological materials are inherently problematic. Biological tissues are either in vivo or not. In either event, the amount of a sample, the repeatablility of a sample, the control and observable characteristics of a sample are nearly impossible to maintain when dealing with biological materials. Moreover, the replication of biological materials, organisms, tissues, or other substances is extremely difficult. Moreover, the variation in conditions cannot be precisely controlled in many circumstances. Providing identical conditions, genetics, and the like in an organism is not a practical mechanism for generating calibration samples.

On the one hand, generating complex sets of physical data, electron counts, currents, voltages, photon counts, and the like may be possible. On the other hand, collection of such detailed data may be impossible. As a practical matter, such collection and analysis can be extremely complex and prohibitively expensive.

Thus, what is needed is a synthetic material that can be generated, manufactured, or otherwise produced by a predictable set of standards, with some processing that can be repeatably controlled, in order to provide a sample for calibrating a scanner. That is, what is needed is a synthetic material or a system of synthetic materials that can be relied upon to produce and maintain over an extended period of time a consistent radiant response when illuminated by a scanner. Accordingly, such synthetic materials may then be used to establish calibration standards that can be transported and verified worldwide.

Moreover, even within the context of a factory, having a stable, repeatable, reproducible, easily manufactured synthetic sample that can be used to calibrate machine-to-machine variations out of the performance of those machines would be extremely valuable. Moreover, some type of field calibration apparatus and method, particularly if including a reliable synthetic material as a sample, would be a substantial advance in the art in calibrating out the day-to-day or time-to-time variations in the output of an individual scanning apparatus and associated processor.

### BRIEF SUMMARY AND OBJECTS OF THE INVENTION

In accordance with the foregoing needs, a system of various apparatus and methods is disclosed herein for calibrating bio-photonic scanning systems. Moreover, synthetic materials have been discovered, formulated, evaluated, and otherwise made available to perform the various calibration functions required of a bio-photonic scanner. For example, mechanisms have been developed for presenting to a scanner certain calibration materials in repeatable structures and positions in order to obtain reliable radiant responses therefrom. Likewise, various compositions for factory and field calibration operations have been developed. For example, a dark cap for returning substantially no radiant response to a scanner, in response to laser illumination, provides for a mechanism to factor out the electrical and electronic artifacts of the machine. Similarly, a white scan sample has been developed that replicates the shape and values of the spectral response of biological tissues, while being reproducible as a simple non-biological chemical composition.

Moreover, materials have been discovered and developed for doping a matrix of material in order to present synthetic mimics of certain molecular structures of interest. For example, carotenoids and other chemical compositions existing in biological tissue appear to contain certain characteristic carbon bond structures. Synthetic materials have been discovered that contain similar bond structures, responsive to illumination by providing a radiant response (e.g. Raman scattering, etc.) similar to that of biological molecular constituents.

Accordingly, a system and method have been developed to implement synthetic materials as calibration samples in order to calibrate scanning systems repeatably. Moreover, the various compositions and apparatus developed and discovered have been implemented successfully in a series of calculations and mathematical manipulations of data in order to process the output of a scanner, normalizing and otherwise neutralizing undesirable or uninteresting characteristics of spectral curves of radiant intensity. Thus, machine-to-machine variations as well as time-to-time variations within a single machine can be factored out, yielding much better signal to noise ratios and much more evident Raman responses. Accordingly, proper calibration apparatus and methods provide for accurate and repeatable utility of bio-photonic scanner.

### BRIEF DESCRIPTION OF THE DRAWINGS

The foregoing and other objects and features of the present invention will become more fully apparent from the following description and appended claims, taken in conjunction with the accompanying drawings. Understanding that these drawings depict only typical embodiments of the invention and are, therefore, not to be considered limiting of its scope, the invention will be described with additional specificity and detail through use of the accompanying drawings in which:

FIG. 1 is a perspective view of one embodiment of an apparatus in accordance with the invention including several mechanisms for presenting scanning samples during calibration processes;

FIG. 2A is a perspective view of the convex side of a dark cap used in calibration in accordance with the invention;

FIG. 2B is a perspective view of the convex side of the dark cap of FIG. 2A;

FIG. 2C is a perspective view of one embodiment of a shield for identifying an “on” condition of a laser and for diffusing laser energy to preclude specular transmission or reflection of coherent light;

FIG. 3A is a perspective view of one embodiment of a precision cap containing multiple samples of synthetic calibration materials for use in an apparatus and method in accordance with the invention;

FIG. 3B is a right side elevation view of a precision cap positioned for calibration of a scanner in accordance with the invention;

FIG. 3C is a side elevation cross-sectional view of an alternative embodiment of a precision cap illustrating the use of an offset in order to provide a low-valued sample using a high-valued material in accordance with the invention;

FIG. 3D is a side, elevation, cutaway view of a dark cap installed on the barrel and window of an apparatus for calibration in accordance with the invention;

FIG. 4 is a perspective view a spring-loaded calibration apparatus in a closed position;

FIG. 5 is a perspective view of the calibration apparatus of FIG. 4 showing the open attachment bracket, corresponding detent, and the use of a spacer to obtain a reduced-value reading for calibration from a single sample;

FIG. 6 is a rear perspective view of the calibration apparatus of FIG. 4 showing the plunger in the drawn position retracting the sleeve and sample away from the barrel of a scanner as appropriate during installation of the calibration mechanism;

FIG. 7 is a rear perspective view of the apparatus of FIGS. 4-6 showing the plunger and handle in the deployed position placing the sleeve and sample toward the window and barrel of a scanner in accordance with the invention;

FIG. 8 is a perspective view of one embodiment of a double-ended sample system for calibration of a scanner in accordance with the invention;

FIG. 9 is a partially cutaway perspective view of a double-ended, double-sample calibration apparatus in accordance with the invention illustrating the sliding mechanisms and retraction handles for positioning the apparatus in a scanner for use during calibration operations;

FIG. 10 is a perspective view of a window and barrel portion of the probe of a scanner, together with the master sample-system and installation thereof during calibration of a scanner using a synthetic mimic material to replicate the radiant response of tissues;

FIG. 11A is a perspective view of a vertically oriented film material illustrating the operation oriented light waves with respect thereto;

FIG. 11B is a perspective view of a horizontally oriented film material illustrating the operation oriented light waves with respect there;

FIG. 11C is a schematic diagram of one embodiment of a lay up of oriented polymeric film typical of those useful in a calibration apparatus in accordance with the invention, typical of a low-valued calibration sample;

FIG. 11D is a schematic diagram of an alternative embodiment of an oriented, polarizing-type, polymeric film of particular utility as a high-valued sample for use in a calibration apparatus and method in accordance with the invention;

FIG. 12 is a schematic diagram illustrating the relationships between synthetic and other non-tissue materials useful in operation of an apparatus and method for calibration in accordance with the invention, including undoped synthetic matrix materials, dopants, with the resulting master samples and selected radiant response characteristics of the foregoing;

FIG. 13 is a chart illustrating schematically the form of an intensity curve of rediant response as a function of wavelength as a result of elastic, fluorescent, and Raman radiant response effects of a subject;

FIG. 14 is aa chart illustrating schematically the Raman scattering effects after normalization for reduction of elastic scattering and fluorescence, as well as dark-scanned electronic artifacts of an apparatus and method in accordance with the invention;

FIG. 15A is chart illustrating schematically a method for selecting a baseline curve fit to match underlying data above which a Raman scattering peak may project in accordance with the invention;

FIG. 15B is a chart illustrating actual normalized and processed scan data from a synthetic master sample, including the fitting of a baseline curve for determination of the characteristic peak desired for calibration processes;

FIG. 15C is a chart illustrating actual data after processing and normalization, fitted with a baseline curve in order to ascertain the value of the characteristic peak for an actual scan of tissue by an apparatus and method in accordance with the invention;

FIG. 15D is a chart illustrating actual data after processing and normalization, and fitted with a baseline polynomial curve, based on a scan of a calibrating sample of the film type in accordance with the invention;

FIG. 16 is a schematic diagram illustrating various material compositions and formats that can be scanned or otherwise evaluated to obtain raw data, radiant responses, or calibration curves, along with a schematic chart for scaling the calibration of an individual scanned result to the scale of a particular standard for scanning results;

FIG. 17 is a schematic block diagram of one embodiment of a process for calibration relying on synthetic or other master samples to obtain unit-to-unit uniformity, as well as condition-to-condition uniformity over time for a scanner and calibration system in accordance with the invention;

FIG. 18 is a schematic block diagram of a process for formulation and use of a master sample for calibration of a scanner in accordance with the invention, applicable to naturally occurring materials dopants as well as fully synthetic matrix and dopant materials;

FIG. 19 is a schematic block diagram of a method for field operation and calibration of a scanner and calibration apparatus and method in accordance with the invention;

FIG. 20 is an alternative embodiment of a schematic block diagram of a method for field operation and calibration of a scanner and calibration apparatus and method in accordance with the invention;

FIG. 21 is a schematic block diagram of a process for making a dopant in accordance with the invention;

FIG. 22 is a schematic diagram of a mill for grinding a dopant in accordance with the invention;

FIG. 23 is a schematic diagram of a process and apparatus for grinding a dopant in accordance with the invention;

FIG. 24 is a schematic block diagram of a process for compounding a calibration composition in accordance with the invention; and

FIG. 25 is a schematic block diagram of a process for making and using a synthetic calibration composition in accordance with the invention.

### DETAILED DESCRIPTION OF THE ILLUSTRATED EMBODIMENTS

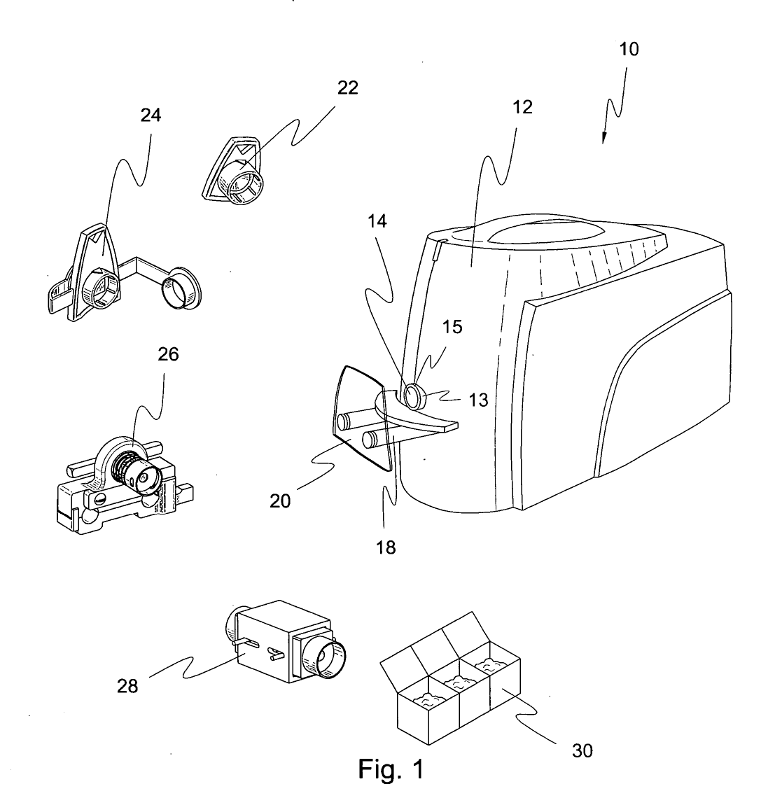

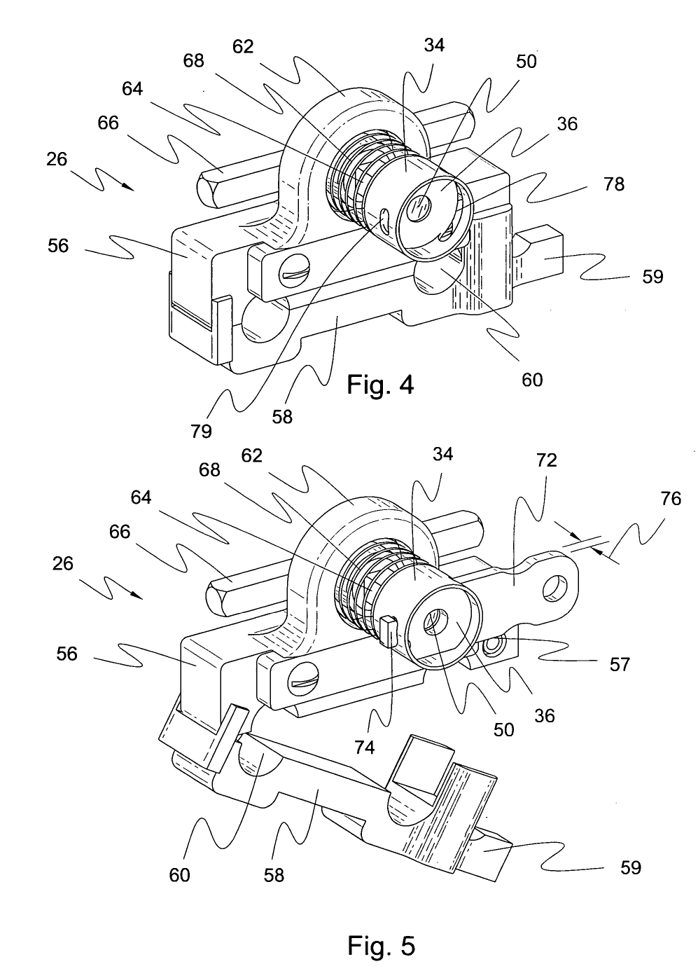

It will be readily understood that the components of the present invention, as generally described and illustrated in the Figures herein, could be arranged and designed in a wide variety of different configurations. Thus, the following more detailed description of the embodiments of the system and method of the present invention, as represented in FIGS. 1 through 25, is not intended to limit the scope of the invention, as claimed, but is merely representative of certain presently illustrated embodiments of the invention.

The various embodiments in accordance with the invention will be best understood by reference to the drawings, wherein like parts are designated by like numerals throughout.

Referring to FIG. 1, an apparatus 10 in accordance with the invention may include a scanning mechanism including a power supply, a light source, such as a laser light source, and a detector. The detector may receive signals including background fluorescence, elastically scattered light (reflections of source light), as well as Raman-scattering light returning to the detector at a wavelength different from that of the incoming illumination beam.

In general, the scanning mechanism will be enclosed within a housing 12, having a barrel 13 penetrating therethrough in order to deliver both illumination and returning detectable beams therethrough. Typically, a barrel 13 may be provided a certain amount of relief or clearance radially between the barrel 13 and the housing 12.

A window 14 mounted in the barrel 13 passes an illuminating beam outward to a subject, and a return “radiant response” back through the window 14 to be received by a detector. For example, a charge-coupled device (CCD) or charge-injection device (CID) may constitute an array of sensors capable of detecting light of various frequencies (e.g. and corresponding wavelengths). Accordingly, a histogram or spectrum of intensities may be displayed over a domain of frequencies or a domain of corresponding wavelengths.

In one embodiment of an apparatus 10 in accordance with the invention, a rest 16 is positioned below and outwardly or in front of the window 14. Supports 18 may extend from the apparatus 10 within the housing 12 to support the rest 16. Accordingly, a hand, arm, or other member of a subject may be positioned on the rest 16 in front of the window 14.

In one presently contemplated embodiment of an apparatus 10 and method in accordance with the invention, a hand of a user is positioned on the rest 16, placing the skin of the palm of the hand against the window 14. In this way, distance effects, as governed by Bier's law are repeatably controlled by the position of the window 14.

A shield 20 may provide several functional features. For example, in one embodiment, the shield 20 is formed of a translucent material shining in response to a beam of light output through the window 14 from the apparatus 10. Light passing through empty space has no mechanism to render it visible outside of the beam itself. Accordingly, as a matter of safety, laser light may be intercepted and scattered by the shield 20. By the same token, a user may be notified that the apparatus 10 is powered up and operating by the visibility of a spot of light illuminating the shield 20.

In various embodiments, the shield 20 may be clear, translucent, textured, or simply otherwise formed to diffuse light randomly. In certain embodiments, the shield 20 may be opaque. In such an embodiment, a user or operator may only see the evidence of a spot of light on the shield 20 between the window 14 and the shield 20. In one presently contemplated embodiment, a diffusion surface is formed on the shield 20 regardless of whether opaque, translucent, or transparent.

In yet another embodiment, a diffusion layer of a material, such as, for example, linen or the like, may be embedded within layers of transparent or translucent polycarbonate in order to provide substantial diffusion. In another embodiment, a simple plastic such as an acrylic or other transmissive polymer may be provided with a dappling or roughened surface on one or both sides in order that the shield 20 provide no specular transmission or reflection of light from the window 14.

Various functional features of the apparatus 10 may be served by a series of accessories such as a dark cap 22 or a dark sample 22 returning no significant beam to the apparatus 10 in response to illumination received from the window 14. Accordingly, a response or radiant response detected by the apparatus 10 after illumination of the dark cap 22 through the window 14 will corresponding to substantially no radiation (e.g. light) of interest.

As a result, illumination of the dark cap 22 through the window 14 results in a signal in the apparatus 10 representing background anomalies, (e.g. electrical or electronic artifacts) of the apparatus 10. In other words, a signal received back into the apparatus 10 in response to a beam illuminating the dark cap 22 provides a signal representing spurious contributions to the apparatus 10 as a direct result of the electrical or electronic artifacts (e.g. errors, background noise, etc.) of the apparatus 10 itself.

Precision samples 24 may be embodied in a film cap 24, sometimes referred to as a field calibration cap 24. The cap 24 may be placed over the window 14 in either a low value or a high value position. That is, precision samples 24 represent a comparatively high value of a return signal into the apparatus 10 and a comparatively low value of a return signal into the apparatus 10. Each may result directly from material samples in the precision cap 24.

That is, the precision cap 24 may be placed in either of two orientations, one hundred eighty degrees apart, in order to expose to the signal (e.g. beam) a material yielding a high or a low radiant response. Both illuminating beam and radiant response propagate through the window 14 from the apparatus 10 and into ti, respectively.

As a direct result, either a high or low value of a radiant response will be transmitted back through the window 14 into the apparatus 10 from the particular materials in the precision samples 24. The high and low values may be a result of the radiant response of materials, the result of distance from the window 14, or both.

A loaded cap 26 provides a mechanism that can be repeatably and stably mounted to the supports 18 in order to provide spring-loaded positioning of a test sample against the window 14. Similarly, a double cap 28 or a test block 28 having spring-loaded caps on both ends, each with a sample, producing a high or low value, is shaped and sized to be positioned between the window 14 and the shield 20. The double cap system 28 provides spring loading in which the sample of interest is urged against the window 14 to provide repeatable registration thereagainst.

Master samples 30 are used primarily in factory calibration. In certain embodiments, master samples 30 may be used for field calibration. The master samples 30 comprise moldable materials that may be temporarily adhered to the window 14 to replicate synthetically the scanning of a bodily member such as a hand. For example, the master samples 30 are structured to be positioned as a putty-like material adhered to the window 14 to produce or appear as neutral background (white scan) results, comparatively low concentrations of molecular compositions of interest and comparatively high concentrations of molecular compositions of interest. The molecular composition of interest is distributed within the putty of the master samples 30 in accordance with the comparative value of the concentration of the molecular constituent of interest desired.

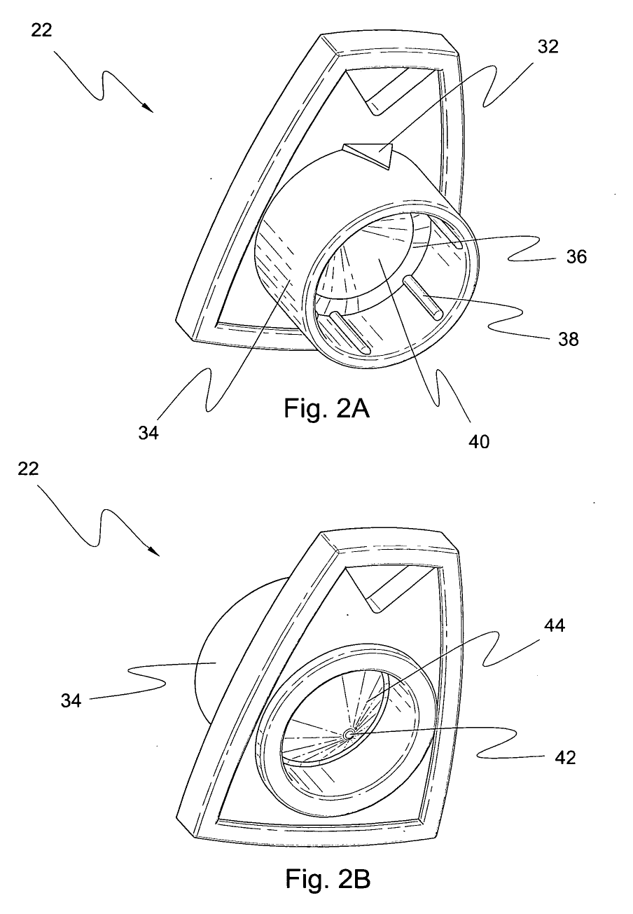

Referring to FIGS. 2A-2C, while continuing to refer generally to FIG. 1, a cap 22, 24 may include, in general, an alignment mark 32. With the dark cap 22, alignment is not particularly significant. However, with respect to the precision samples 24, at least certain embodiments thereof, alignment may be a significant variable and the witness mark 32 or alignment mark 32 may assist in providing precise alignment of the precision samples 24.

Nevertheless, in the dark cap 22, a sleeve 34 fits snugly over the barrel 13, registering a shoulder 36 against the window 14 thereof. That is, the shoulder 36 provides a registration surface 36 that fits against the face 15 of the barrel 13. Typically, the face 15 and the window 14 may be substantially flush with one another.

Shims 38 or spacers 38 provide grip or a snug fit between the barrel 13 and the sleeve 34. As a practical matter, the sleeve 34 may distort to a certain extent as a result of the shims 38 contacting the barrel 13. Accordingly, deflection of the barrel 34, the shims 38, or both elastically provides the force to keep the dark cap 22 snugly fitted against the face 15, and comparatively immovable with respect to the barrel 13.

In operation, the dark cap 22 includes a black sample 40. In the illustrated embodiment, the black sample 40 is simply a concave, dark, surface 40. In certain embodiments, a light trap, collimated, black, light trap, a black fabric, or the like may serve as the black sample 40. The angled surface and the black material of the black sample 40 disperse away from the window 14 the illumination proceeding out of the window 14, in order that substantially no radiant response be returned back through the window 14 into the apparatus 10.

Accordingly, the dark cap 22 absorbs, deflects, and otherwise disperses the signal proceeding from the window 14 in order to provide to the apparatus 10 a “dark” reading reflecting nothing more than the electrical and electronic artifacts of the apparatus 10. The reading or any signal detected or recorded by the apparatus 10 in response to the illumination of the dark cap 22 is actually only an artifact of background and error effects endemic to the apparatus 10. Accordingly, the dark cap 22 may be used to provide a background signal to be deducted from scanning readings in order to factor out the electrical and electronic artifacts of the apparatus 10.

Opposite the concave surface 40 constituting the black sample 40, a convex surface 41 proceeds to a vertex 42. The convex surface 41 may serve as a dark sample 41. Nevertheless, since manufacturing processes typically provide easily for a sharp vertex 42 on the interior (concave) surface 40, but do not provide a precision point of a convex surface 41, the vertex 41 typically interferes with proper operation of the dark cap 22 if used as the dark surface 41.

Referring to FIG. 2C, the shield 20 may be illuminated by a beam of light through the window 14 resulting in a light spot 43. The light spot 43 or light region 43 may be seen from either major surface of the shield 20. If the shield 20 is translucent or transparent, the light spot 43 may be viewed from substantially any significant angle with respect to the apparatus 10. However, if the shield 20 is opaque, then the light spot 43 will typically only be seen from a position viewing the surface of the shield 20 directed toward the apparatus 10.

Nevertheless, in one embodiment of an apparatus and method in accordance with the invention, the shield 20 may be formed of multiple layers 44. In one embodiment, a single layer 44a of a translucent material, such as acrylic, polycarbonate, polystyrene, or the like may serve as the bulk material of the shield 20. The surfaces 44d, 44e of the shield 20 may themselves be textures on an otherwise transparent material in order to provide diffusion dispelling any specular reflection or transmission of a beam from the light spot 43.

In one presently contemplated embodiment, a layer 44a, together with a layer 44c, may sandwich a diffusion material 44b therebetween. For example, polycarbonates are virtually unbreakable. Accordingly, two layers 44a, 44c of polycarbonate may be molded as a unit embedding a layer 44b, or laminated together with a layer 44b of linen therebetween. In the illustrated embodiment, the layer 44b intermediate the layers 44a, 44c may provide a substantial scattering effect precluding the passage of any substantial specular light.

As a practical matter, laser powers within the apparatus 10 may be selected to be sufficiently low as to cause no tissue damage, particularly ocular damage, even accidently. Nevertheless, the shield 20 serves as both a warning that the apparatus 10 is powered up and operating, as well as a protection against any over exposure of eyes to its modest intensity. The highly diffusive shield 20 may substantially inhibit any specular transmission or reflection of light impinging at the light spot 43 from the window 14. With or without the intermediate diffusion layer 44b, the surfaces 44d, 44e may still be provided with roughening or texturing as a scattering mechanism.

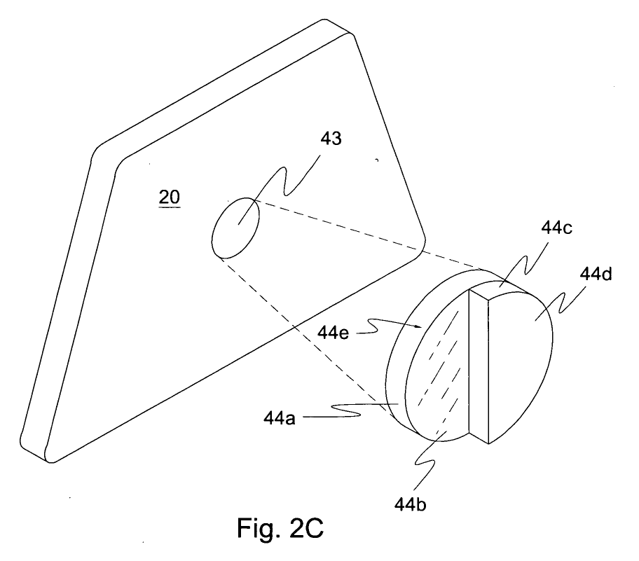

Referring to FIGS. 3A-3D, while continuing to refer generally to FIGS. 1-3, a precision cap 24 may include a witness mark 32 or alignment mark 32 in order to orient the cap 24 circumferentially with respect to the barrel 13. The precision cap 24, or more properly the sample materials 50 incorporated therein, may be orientation sensitive. Rotation of the precision cap 24 with respect to the barrel 13 may alter the radiant response reading detected by the apparatus 10 in response to illumination of the sample material 50 by a light beam.

Typically, two sleeves 34 on opposite sides of the precision cap 24 are provided, each with shims 38 for a snug fit against the barrel 13. The shoulder 36 serves to register the cap 24 against the face 15 of the barrel 13.

In the illustrated embodiment, dust covers 46 fit within and against the sleeves 34 to protect against scratching, accumulation of debris, and the like. In certain embodiments, the dust covers 46 may be connected to the precision cap 24 by an arm 48, which arm 48 may be integrally molded with the basic structure of the precision cap 24.

The foot 52 or feet 52 formed as part of the precision cap 24 are configured to fit snugly against the rest 16. Accordingly, the foot 52 assists in maintaining alignment of the sample material 50 with respect to the window 14. The feet 52 tend to urge the cap 24 into the proper orientation. Meanwhile, the witness mark 32 may assure that alignment of the cap 24 comports with the desired position with respect to the barrel 13.

In one presently contemplated embodiment, an aperture 54 receives a tether 55 anchored to the apparatus 10. For example, the tether 55 may tie to the supports 18 in order that the precision sample 24 may not be removed from nor substituted away from the apparatus 10.

The sample materials 50 may be configured to provide high and low respective values of a radiant response in consequence of illumination by a light beam from the window 14. Opposite sides of the sample 24 provide sleeves 34 surrounding samples 50. One sample 50 provides a comparatively lower reading, and the other sample 50 provides a comparatively higher reading.

In one embodiment, one corner is truncated from the sample 50 and a corresponding relief is formed in the shoulder 36. Accordingly, the sample 50 may only be positioned in one single orientation framed by the shoulder 36. Thus, the sample 50 is precisely aligned with the structure of the precision cap 24, and the precision cap is oriented by the feet 52 against the rest 16 in order to provide precise orientation with respect to the window 14.

It is known that carotenoids return light according Raman scattering principles as a radiant response to illumination by certain light. For example, light on the order of 473 nanometers in wavelength excites certain carbon bonds within carotenoids. It has been discovered that similar carbon bonds, and particularly a double carbon bond exists in certain oriented polymeric films. Accordingly, when the samples 50 are formed of particular types of polymeric films, an excitation of carbon bonds by light of suitable frequencies, such as laser light of approximately a 473 nanometer wavelength, Raman scattering at 510 nanometers wavelength results.

Accordingly, the samples 50 maybe formed of a comparatively stable, nonperishable, synthetic material, rather than a naturally occurring or biological tissue material. For example, prior art apparatus such as those developed by Gellermann et al. (see U.S. Pat. No. 6,205,354 B1, issued Mar. 20, 2001 to Gellermann et al., incorporated herein by reference) could rely on actual biological tissue destructively obtained. Comminuted tissue samples from cadavers can provide materials for testing. By contrast, the samples 50, formed of a synthetic material providing a suitable response will provide much better repeatability, much more uniformity, as well as a substantially unlimited supply of uniform samples 50.

Referring to FIG. 3B, a precision sample 24 or precision sample cap 24 may fit snugly against the barrel 13 of an apparatus 10. The housing 12 may be relieved in order to receive the sleeve 34 around the barrel 13. In one presently contemplated embodiment, the sample 50 is adjusted snugly against the window 14 of the apparatus 10. The foot 52 fits against the rest 16 or plate 16, orienting the cap device 24. The cap 24 may be reversed to exchange a high valued sample 50 for a low valued sample 50 over the window 14.

Referring to FIG. 3C, in one variation of an embodiment of a precision cap 24 in accordance with the invention, a low valued sample 50a may be set into the structure of the cap 24, providing an offset distance 55. That is, the materials for films forming the samples 50a, 50b are not only sensitive to rotation or orientation of their oriented polymeric fibers, but are governed to some extent by Bier's law. The distance 55 or offset 55 of a sample 50a away from a window 14 will affect the radiant response of the sample 50a to a beam of light from the window 14.

Accordingly, the offset 55 may be selected and calculated to provide a particular decrease in the radiant response of the sample 50a. By the same token, a high valued sample 50b may remain flush with the window 14, or positioned with a different offset 55. Thus, a single actual material with its single value for a radiant response may actually serve to provide different radiant responses by simply positioning a low value sample 50a at a deeper or more distant offset 55 with respect to a higher valued sample 50b.

Referring to FIG. 3D, positioning a dark cap 22 against a window 14 exposes an angled and concave surface 40 exposed to the beam from a window 14. Accordingly, the beam is dispersed rather than returning back through the window 14 as a radiant response. Accordingly, the radiant response of a dark cap 22 is substantially a null response, resulting in data corresponding to the background value corresponding to electrical and electronic artifacts (e.g. errors, noise, etc.) of the apparatus 10.

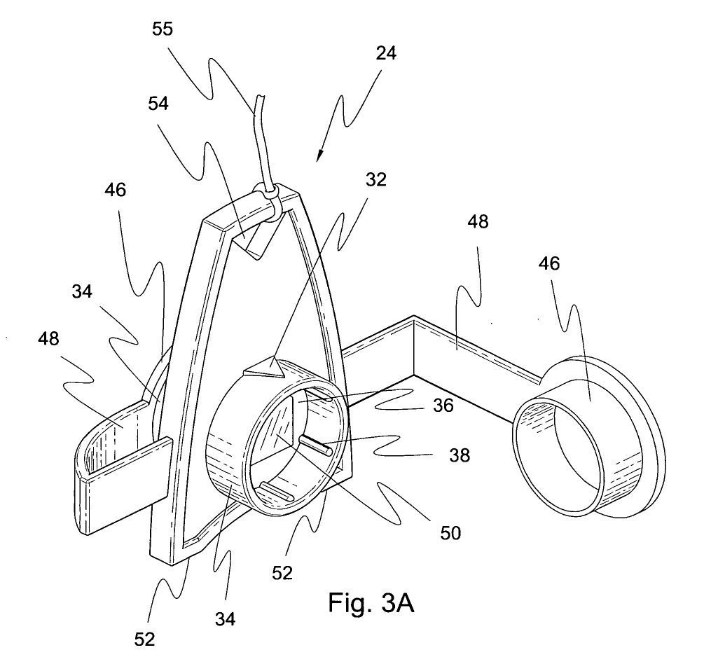

Referring to FIGS. 4-7, one embodiment of a loaded cap 26 or self-loading cap 26 may include a mount 56 sized to fit over the supports 18. A matching bracket 58 closes against the mount 56. An operator, moving the handle 59 toward the mount 56 engages a detent 57 snugly holding closed the bracket 58 against the mount 56.

The self-loading cap 26 includes a sleeve 34 that can slide with respect to the mount 56 toward the face 15 of the barrel 13. Thus, the shoulder 36 registers a sample 50 with respect to the window 14 in order to achieve the proper and repeatable radiant response. Typically, the supports 18 are received into apertures 60 formed between the mount 56 and the bracket 58. The mount 56 may thus be adjusted to position a receiver 62 sufficiently close to the window 14 to properly place the sleeve 34 and shoulder 36 with respect to the window 14 and face 15 of the barrel 13.

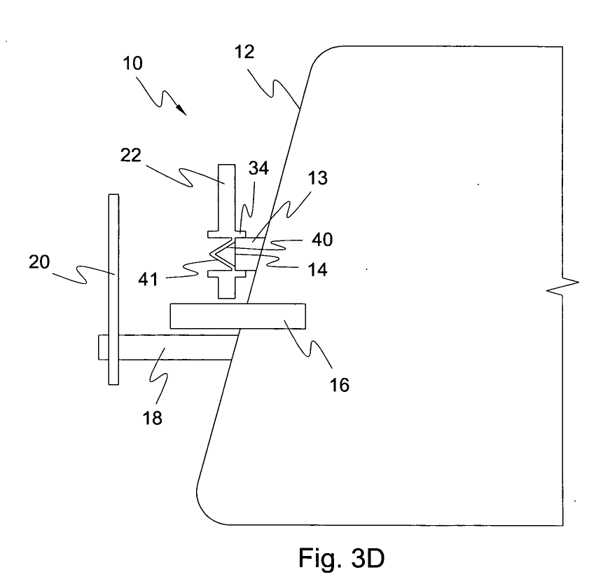

In one presently contemplated embodiment, the receiver 62 receives a plunger 64 penetrating therethrough and positionable by a handle 66. The handle 66 may be drawn back, compressing a spring 68 between the sleeve 34 and the receiver 62. The plunger 64 is detained by a detent (not shown) operating between the receiver 62, and the plunger 64. Accordingly, the sleeve 34 and shoulder 36, along with their supported sample 50 are effectively retracted away from the window 14. In such a position, as illustrated in FIG. 6, the supports 18 of the apparatus 10 may be positioned within the aperture 60 to place the sleeve 34 proximate the barrel 13.

Upon urging the handle 66 toward the barrel 13 and included window 14, the detent is overcome, the spring 68 urges the sleeve 34, shoulder 36, and included sample 50 forward toward the window 14. The shoulder 36 registers against the face 15. Registration of the shoulder 36 against the face 15 positions the sample 50 with respect to the window 14.

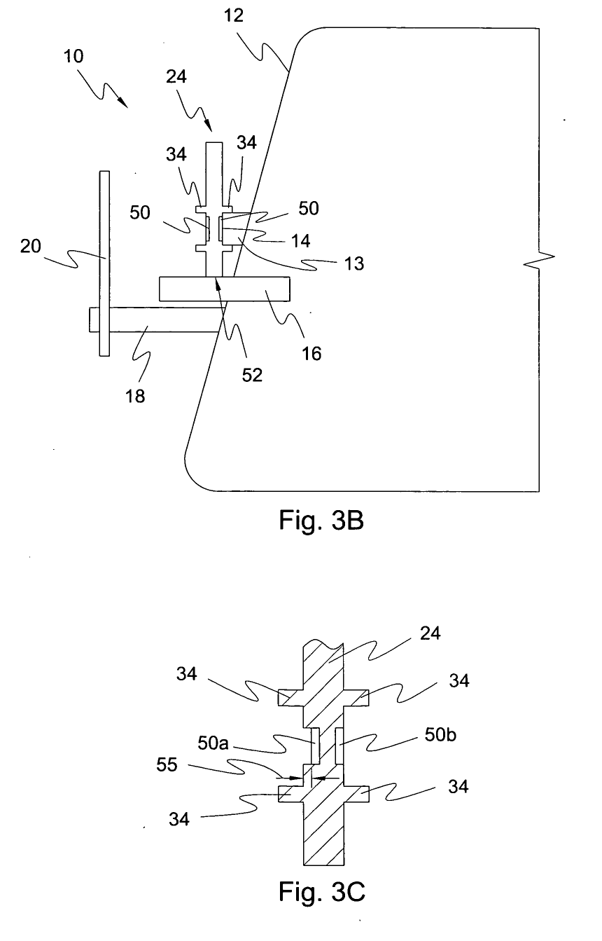

In one currently contemplated embodiment, a spacer 72 extends laterally or radially through the sleeve 34 extending out at an end 74. The spacer 72 is perforated to expose to view the sample 50. However, as illustrated in FIG. 5, the thickness 76 or standoff distance 76 provided by the spacer 72 spaces a sample 50 a distance 76 away from the window 14 of the barrel 13. The offset 76 is calculated and tested to provide a sufficient decrease in the radiant response of the sample 50 to illumination from the window 14 to provide a “low value” of radiant response.

The entry aperture 78 for the spacer 72 may be larger than the exit aperture 79. Thus, the end 74 may be smaller in cross section than the bulk of the spacer 72. Accordingly, the spacer can be registered within the sleeve 34 in order to provide a stable positioning of the perforation exposing the sample 50. The plunger 64 is advanced through the receiver 62 by pressing the handle 66 toward the barrel 13 and enclosed window 14. The plunger 64 advances the sleeve 34, shoulder 36, and sample 50 toward the window 14. Likewise, the spacer 72 positions the shoulder 36 further from the window 14, acting as the shoulder 36 itself 72.

In such a circumstance, the spring 68 urges the sleeve 34 and shoulder 36 with the included sample 50 forward toward the face 13 and window 14 to the extent possible. Thus, a repeatable registration of the sample 50 with respect to the window 14 results. Meanwhile, the spacer 72 provides a second and lower radiant response from the sample 50 by virtue of the distance differential in positioning of the sample 50 with respect to the window 14.

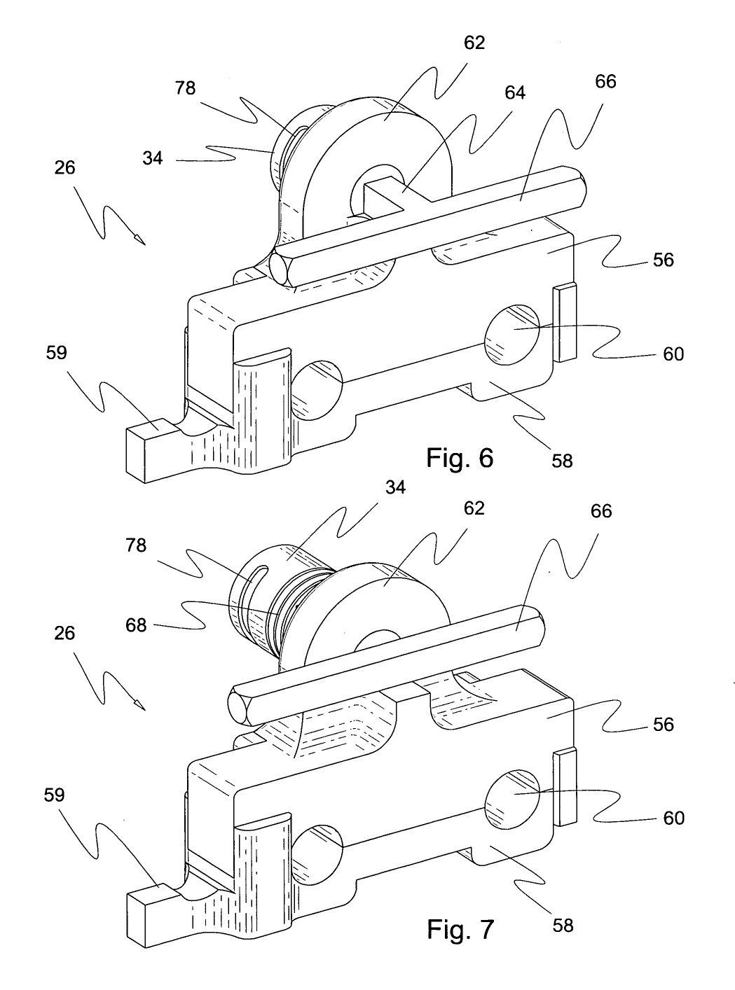

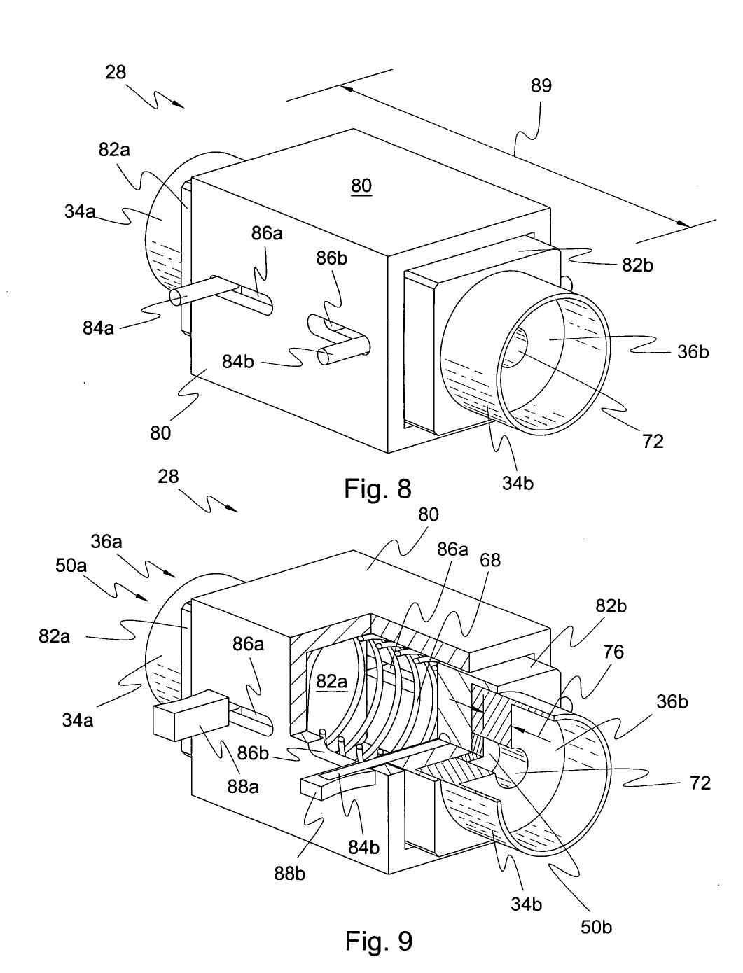

Referring to FIGS. 8-9, a double cap 28 or a double-ended cap 28 may include a frame 80 fitted with opposing slides 82a, 82b from the ends thereof. The slides 82a, 82b may each carry thereon a respective sleeve 34a, 34b. Each sleeve 34a, 34b may present a respective sample 50a, 50b offset by an appropriate spacer 72 as needed. In operation, the spring 68 urges the slides 82 apart.

Handles 84 operating in slots 86 through the wall of the frame 80 secure to the slides 82 in order to retract the slides 82. That is, for example, the handles 84, or the handles 84 provided with plates 88 or thumb plates 88 can be drawn together by a user in order to retract the slides 82a, 82b with their respective sleeves 34a, 34b. In this way, the effective length 89 of the apparatus 28 or cap system 28 may be reduced in order to fit easily between the window 14 or face 15 and the shield 20.

Accordingly, the frame 80 is positioned conveniently on the rest 16 or deck 16 under the window 14. Upon release of the handles 84 by a user, the spring 68 urges the respective slides 82 and associated sleeves 34 apart. One sleeve 34a, 34b will contact the shield 20, while the opposite sleeve 34b, 34a will surround the barrel 13 and position the respective shoulder 36 against the face 15 and included window 14. Thus, a snug fit of a shoulder 36 with respect to a window 14 will position a sample 50 properly for returning the designated, calibrating, radiant response through the window 14 in response to illumination received from the apparatus 10 through the window 14.

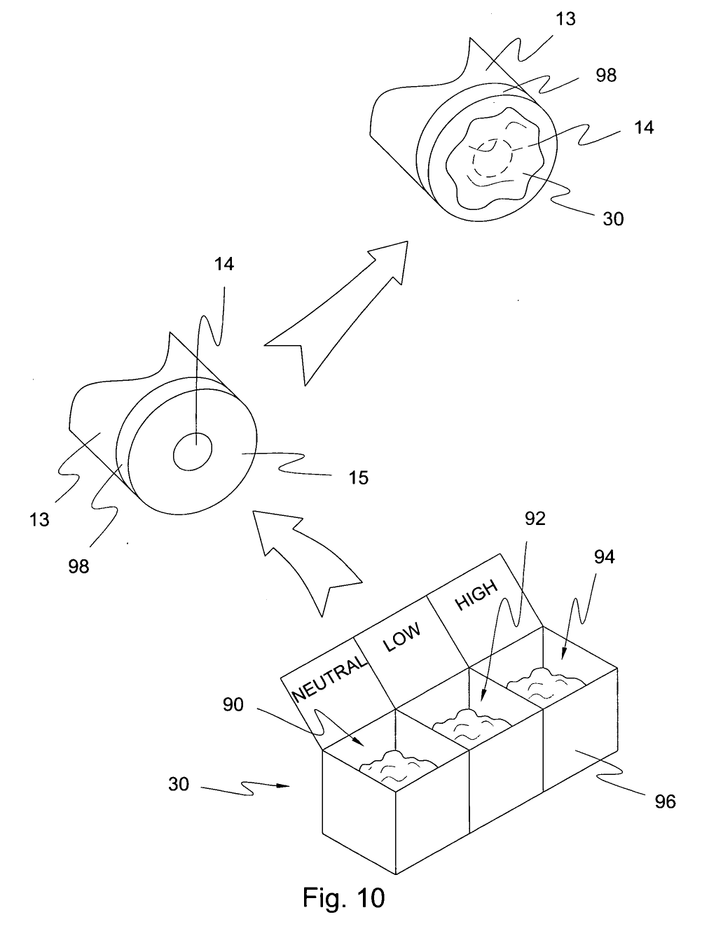

Referring to FIG. 10, a master sample 30 may actually include a neutral sample 90, a low-valued sample 92, and a high-valued sample 94. Having these three samples 90, 92, 94 in a properly labeled case 96 provides a set of standards by which a factory calibration can substantially neutralize machine-to-machine variations in performance. That is, the master sample 30 or sample set 30 provides calibration standards to assure that each apparatus 10 produced will provide a substantially equivalent reading on the same sample material.

A master sample 30 may be adhered to the face 15 and window 14 directly. Typically, the window 14 is secured to or within the barrel 13 by some mechanism, such as a collar 98 or other internal registration mechanism. Accordingly, the window 14, itself, determines the actual positioning of the sample 30.

The thickness of the sample 30 should be sufficient to preclude any transparency or translucence. Likewise, the sample 30 should cover the window completely to preclude ambient light. By the same token, a hand, arm, or other member of a subject may likewise be placed in direct contact with the window 14 in order to provide a proper preclusion of ambient light as well as distance registration of the subject for testing.

Applicants have discovered that the master sample 30 may be effectively formed of a polymer composition. In one presently contemplated embodiment, a material identified as Dow Corning 3179 dilatant compound has been found highly effective to replicate certain properties of human tissues extremely efficaciously. In general, material comprising silicone oil cross linked by boric acid has been found very effective to provide a similar reflectance or elastic light scattering, as well as similar fluorescence, compared to those detected from human skin.

In one presently contemplated embodiment, the master sample set 30, and in particular the neutral sample 90 or white scan sample 90 may include dimethyl siloxane. These are hydroxy-terminated polymers with boric acid. In addition, silica as crystalline quartz may be added to the composition, as well as a proprietary thickener. The thickener is identified by the manufacturer brand name as thixotrol ST.

Other silicone compositions included include polydimethylsiloxane as well as a trace of decamethyl cyclopentasiloxane. A similar amount of glycerine and titanium dioxide may be added to the composition.

In one presently contemplated embodiment, the master sample 30, and particularly the matrix that forms the neutral sample 90 contains approximately 65 percent dimethyl siloxane, 17 percent silica, nine percent thickener, four percent polydimethylsiloxane, one percent decamethylcyclopentasiloxane, one percent glycerin, and one percent titanium dioxide. The matrix material that forms the neutral sample 90 may be characterized as a viscoelastic material. That is, the material 90 responds elastically in response to high rates of strain (e.g. impact), and responds as a liquid in response to comparatively very low rates of stress and strain (e.g. its own weight).

In order to provide the low-valued sample material 92 and the high-valued sample material 94, a doping agent or dopant may be mixed into the neutral sample 90. Naturally occurring or “organic” materials from biological sources have been found effective. For example, foodstuffs containing high values of carotenoids may be comminuted (e.g. pulverized, ground, etc.) and mixed into the matrix material 90. Tomatoes, carrots, vegetables, fruits, and the like containing suitable values of carotenoids can be substantially mixed or dissolved within the matrix 90 in order to produce the samples 92, 94.

Applicants have also discovered that synthetic materials exhibiting the carbon bonding behaviors of carotenoids may also be ground, milled, or otherwise comminuted and dispersed into the matrix material 90 in order to produce the low valued sample 92 and high valued sample 94. For example, after a factory calibration using the master sample 30 comprising a white scan sample 90, a low-valued sample 92, and a high-valued sample 94 can calibrate out the machine-to-machine variations of the apparatus 10.

That is, different concentrations of a micropulverized or comminuted synthetic material having a radiant response characteristic to mimic carotenoids may serve as a highly stable, repeatable, reproducible sample for the high valued material 94 and the low valued material 92. The concentrations of such synthetic dopants within the matrix 90 may be adjusted in order to provide a suitably low value for the low valued or “low” material 92, and a suitably high value for the high valued or “high” material 94.

It has been found that certain materials made from polyvinyl alcohol operate to perform this doping function. For example, K-type polarizer film materials are formed of long polymers called oligomers. Such materials are used as polarizing filters. They may be formed on substrates as polarizing films. These materials are formed of a molecularly oriented polyvinyl alcohol containing oriented block segments of polyvinylene and polyvinylalcohol. In particular, sheets of such K-type polymeric materials include polyvinylalcohol/polyvinylene block copolymer materials where the polyvinylene blocks are formed by molecular dehydration of a sheet of polyvinylalcohol.

This sheet then forms a uniform distribution of light-polarizing molecules of polyvinylalcohol/polyvinylene block copolymer material varying in length. The length is typically a varied value of a length and is characterized by a large number, n, of conjugated repeating vinylene units of the polyvinylene block of the copolymer, in ranges from two to twenty-four.

The concentration of each polyvinylene block tends to absorb wavelengths ranging from two hundred to seven hundred nanometers, and remain substantially relatively constant. The film is identified by its spectral dichroic ratio or R(D). The dichroic ratio increases with the increasing length n of the polyvinylene blocks. Thus the polyvinylene block concentration and the degree of orientation of the molecules result in a photo-optic dichroic ratio on the order of at least about forty-five. Such materials are produced by various manufacturers, and are disclosed in U.S. Pat. No. 5,666,223 incorporated herein by reference.

Applicants have discovered that grinding such materials into a very finely pulverized size results in a dopant that can be satisfactorily distributed within a matrix 90 of dilatant compound in order to provide suitable low value materials 92 and high value materials 94. Dopant may be ground from the treated face of a CAB/K-type material. K-type material by itself may be milled, sanded, or otherwise comminuted to serve as a dopant. In one presently contemplated embodiment, a four hundred grit emery paper having a closed face in order to prevent impurities has been used to grind the dopant material from integrated solid sheet form into a powder. The powder appears to form as elongated crystals. The powder serves adequately when segregated to pass through a number 200 sieve as known in the chemical arts. Particles in larger sizes such as a number 100 sieve, or even 50 are possible, but uniformity of size and dispersion seem to enhance uniformity of results.

The value of a low value and a high value sample 92, 94 may be ascertained by testing a wide range of samples of human subjects. Thereafter, a suitable amount of dopant may be added to the matrix 90 in order to provide a suitable low value material 92 and a suitable high value material 94 representing comparatively high and comparatively low ranges of radiant response corresponding to those of human tissues in vivo.

The master samples 30 provide great utility inasmuch as they can be repeatably compounded from synthetic materials to provide very stable results. To the extent that radiation (e.g. light) may affect the molecular bonds in a material relied upon for testing and calibration, the matrix 90 may be molded to expose different particles. That is, the matrix 90 being a moldable plastic or viscoelastic material may be molded or kneaded in order to thoroughly and evenly disperse the selected amount of dopant.

By the same token, to the extent that a dopant material may alter its chemical structure as a result of continued or prolonged radiation, the master samples 30 may be kneaded in order to redistribute dopant and provide a continuing, substantially constant value of the radiant response therefrom in response to illumination from the window 14 of the apparatus 10.

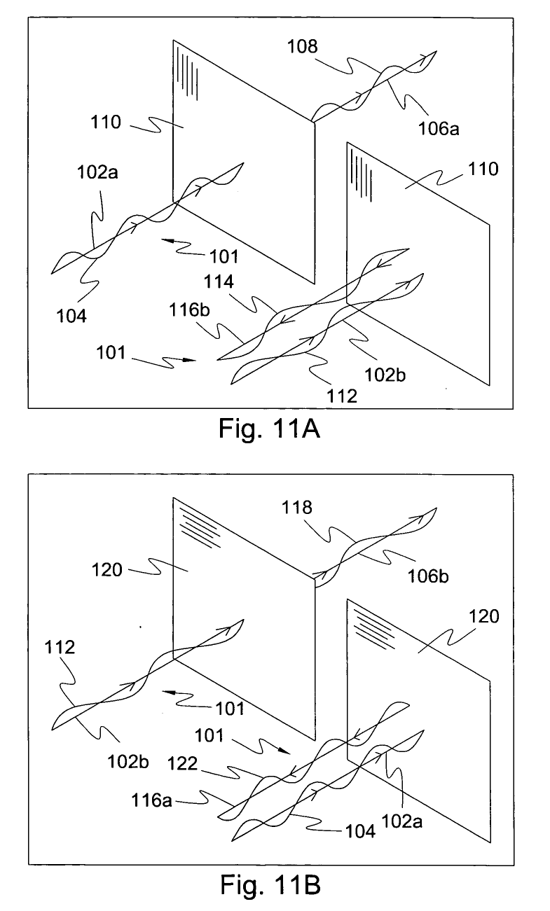

Referring to FIGS. 11A-11D, such a film material may serve directly as a day-to-day calibration material in the precision cap 24. Applicants have discovered that scanning individual human beings or tissue samples presents too many issues of safety, scaling, and the like, and too broad and uncontrollable variations in the performance of the apparatus 10. A day-to-day calibration with synthetic materials is still appropriate in order to work out various conditional variations. For example, temperatures, humidity, electronic drift, and the like may alter the operation of the components of an apparatus 10. Accordingly, with each startup of a scanning session, or even after an extended period within a single scanning session, calibration of the apparatus 10 may be appropriate.

Tethered to each apparatus 10 is a precision cap 24 used to calibrate with respect thereto the apparatus 10. Thereafter, as the machine ages, conditions change, and so forth, the apparatus 10 may be recalibrated such that it can output numerical values resulting from scans in a predictable, consistent, repeatable manner.

In one embodiment, the samples 50 embodied in the precision cap 24 may actually operate as circular polarizers. Circular polarizers combine linear polarizers with quarter-wave retarders. Unpolarized light passes through a linear polarizer and is oriented in one direction. It then passes through a quarter-wave retarder and becomes circularly polarized. That is, it tends to “spin” in a helical fashion. Upon contacting a surface, it may be reflected, returning from the reflecting surface in a reverse helical direction. Return light is limited in its ability to pass back through the initial polarizer. After being linearly polarized with a new orientation at ninety degrees to the transmission axis of the polarizer, the beam has effectively met a barrier similar to two linear polarizers oriented at right angles.

In order to protect the material that provides the radiant response to incoming light, protective coatings may be applied. In certain embodiments, the film of the samples 50 may include a thin sheet of polyvinylalcohol (PVA) aligned and stretched in a sandwich configuration between supporting sheets of cellulose acetate butyrate (CAB).

Referring to FIG. 11A, light may impinge along an incoming axis 102 (for example, axes 102a, 102b) as a vertically oriented wave 104. As a result of impinging upon an oriented film 110 with the orientation as illustrated, the vertical wave may pass through along an outgoing axis 106. Thus, the passed wave 108 passes through a sheet 110 oriented in the same direction as the incoming wave 104. One may note that the orientation of the wave 104 that will be passed through the film 110 is actually orthogonal to the orientation of the actual strands of polymer or oligomer that form the film 110.

By the same token, the vertically oriented film 110, when approached by a wave 112 horizontally disposed along an incoming axis 102b will be absorbed or reflected from the film 110, resulting in a reflective wave 114 traveling along the axis 116b. A beam 101 of nonoriented light will include light 101 in possibly all orientations. Upon impingement of the beam 101 upon a vertically oriented film 110, vertical components 104 pass through as the passed wave 108, whereas horizontal components 112 are absorbed or reflected back as reflected waves 114.

Referring to FIG. 11B, an impinging horizontal wave 112 along an axis 102b will result in a passed-through wave 118 along a retreating axis 106b after impinging on the horizontally oriented film 120. However, just as the beam 101 is effectively “split” by a vertical polarizing film 110, the horizontal polarizing film 120, when impinged upon by a beam 101 or the vertical components 104 thereof along an incoming path 102a, will absorb or return vertical components as a reflected wave 122 along a path 116a and may provide other radiant responses thereto. As a practical matter, the paths 102a and 116a may be identical if a film 120 is oriented precisely normal to an incoming ray 101 of coherent light. Other rules of reflection apply otherwise.

Referring to FIG. 11C, in one presently contemplated embodiment, a film identified as a KNCP35 circular polarizing filter available from 3M Company, provides a polyvinylalcohol (PVA) layer 124a that is partially oriented, thus operating as a quarter-wave polarizer. Thereafter, a layer 124b of an optically clear cellulose acetate butyrate (CAB) and substrate may be followed by another layer 124c of polyvinylalcohol and polyvinylene cross linked with boric acid stretched by an order of magnitude in order to provide orientation.

Plastics stretched in a first direction will pass light oriented in a direction orthogonal to the direction of linear orientation of the long molecules. As the light beam 101 from the scanning apparatus 10 passes through a dichroic filter, the light is not polarization controlled. Nevertheless, the films 110, 120 that serve as the samples 50 will be polarity-sensitive.

Accordingly, the precision cap 24 will behave differently on each apparatus 10. The polarization or whatever the polarity might be of a light beam 101 emanating from the window 14 of the apparatus 10, will simply be tolerated in one presently contemplated embodiment. Nevertheless, the orientation of the sample film 50 in the precision sample 24 used in day-to-day calibrations processes must be repeatable.

Therefore, the material 50 may be oriented, and that orientation will be fixed with respect to the cap system 24, and oriented in accordance therewith. Similarly, the cap 24 will be oriented by the feet 52 on the deck 16 or rest 16 under the window 14. In some embodiments, the film of FIG. 11C may actually be laminated onto a substrate. For example, a base 124d may actually be formed of glass or the like. In alternative embodiments, no base 124d is present. Rather, the CAB material of the intervening optically clear layer 124b may serve as the structural substrate therefor.

In certain embodiments, a high and low film samples 50 may simply be made of a single film composition, and positioned at different locations in order to provide comparatively higher and lower radiant responses (e.g. readings). In other embodiments, different films may be used for the high and low valued samples. For example, a film known as HR-type may actually provide a comparatively low value of radiant response. Such a film is a polyvinylalcohol/polyvinylene having a particular set of double bonds of carbon atoms. This material is also doped with iodine and is often used in infrared spectroscopy.

By contrast, a film known as KNCP35 and other similarly constituted “K-based” films available from 3M Company provide comparatively high radiant response values when exposed to illumination by a beam 101 from the window 14 of the apparatus 10. The KNCP35-type of film operates as a circular polarizing, sandwicHR type of film as described hereinabove.

Low values of scanner outputs (e.g. intensity, score, etc.) for calibration may be obtained with HR-type films 110, 120. Such films include layers of PVA 124a, CAB 124b, and K-type film 124c as shown in FIG. 11C, with or without the base 124d. On the other hand, high value of scanner output for calibration will result from films 110, 120 such as that of FIG. 11D wherein K-type film is bonded to a shielding layer of optically clear CAB.

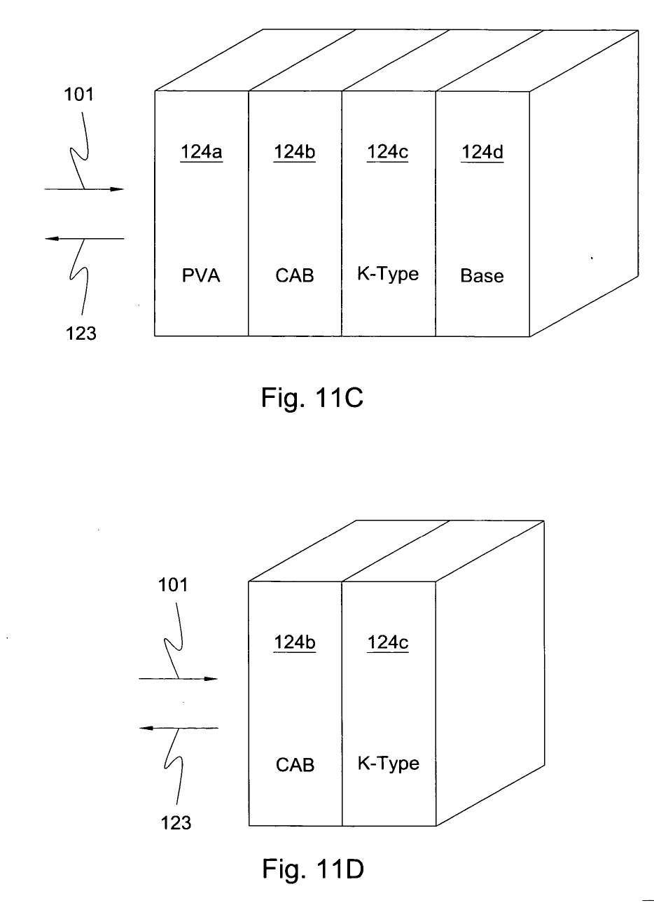

Referring to FIG. 12, applicants have observed that non-tissue materials 125a when exposed to a beam 101 from a window 14 of an apparatus 10 may result in comparatively clearer and well-defined shapes 126a. An area 127a of intersection may place either region 127b, 127c inside the other 127c, 127b or offset as illustrated. The intersection 127a may be substantially less than the area or size of a source envelope 127b representing the area of illumination from the beam 101 proceeding from the window 14. Likewise, the area 127a of intersection may be substantially less than, and misaligned with the area 127c of the detector envelope 127c.

That is, the center of the region illuminated by the source, the source envelope 127b, and the region that is “read” by the detector in the apparatus 10, the detector envelope 127c may be misaligned. The area 127a may therefore be insufficient to be representative of the real radiant response of a sample, calibration material 30, or the like.

By contrast, human skin and the undoped matrix 125b (the neutral material 90 from the master sample set 30) provide a blooming response 127b. Rather than the clearly defined envelopes 127b, 127c that occur in many other materials, human skin as well as the undoped matrix material 125b of the neutral sample 90 or white scan sample 90 of the master sample 30 provide a blooming shape 126b. This blooming shape 126b may be thought of as an enlarged area of radiant response, reflection, scattering, and the like in a highly spread shape 126b. The blooming shape 126b or effect 126b results in a much better intersection 127a between the source envelope 127b and the detector envelope 127c.

Thus, the undoped matrix 125b (e.g. material 90) represents comparatively accurately the behavior of human skin, absent the Raman scattering effect due to carotenoids or other materials containing similar carbon bonds. A curve 126e reflecting the elastic scattering portion and the fluorescence of skin, may be achieved by using the undoped matrix 125b as a calibration sample.

In contrast, dopant materials 125c, such as naturally occurring materials or synthetic materials having the proper carbon bond structures to mimic the behavior of carotenoids or other molecular structures of interest provide a curve 126c identified as a Raman response. Thus, the peaks, and particularly the highest peak typically found at 510 nanometers wavelength, result from illumination of a dopant 125c by the light illuminating test samples 30, 50 from the window 14 of the apparatus 10.

Applicants have discovered that compounding a dopant 125c into the matrix 125b provides the master samples 30 capable of substantially replicating the behavior of human skin reliably and repeatably. The curve 140 of intensity as a function of wavelength obtained by illuminating and reading (e.g. scanning) the master sample 30 provides the full spectral profile 140 expected from the skin of a subject. The neutral sample 90 comprised of the undoped matrix 125b provides a curve 126e capable of identifying, and therefore neutralizing out, the effects of elastic scattering of illuminating light, as well as the skin's natural fluorescence. Meanwhile, different concentrations of doping in the low value sample 92 and the high value sample 94 of the master sample 30 provide comparatively different curves 140 and particularly the Raman response curves 126c contributing thereto.

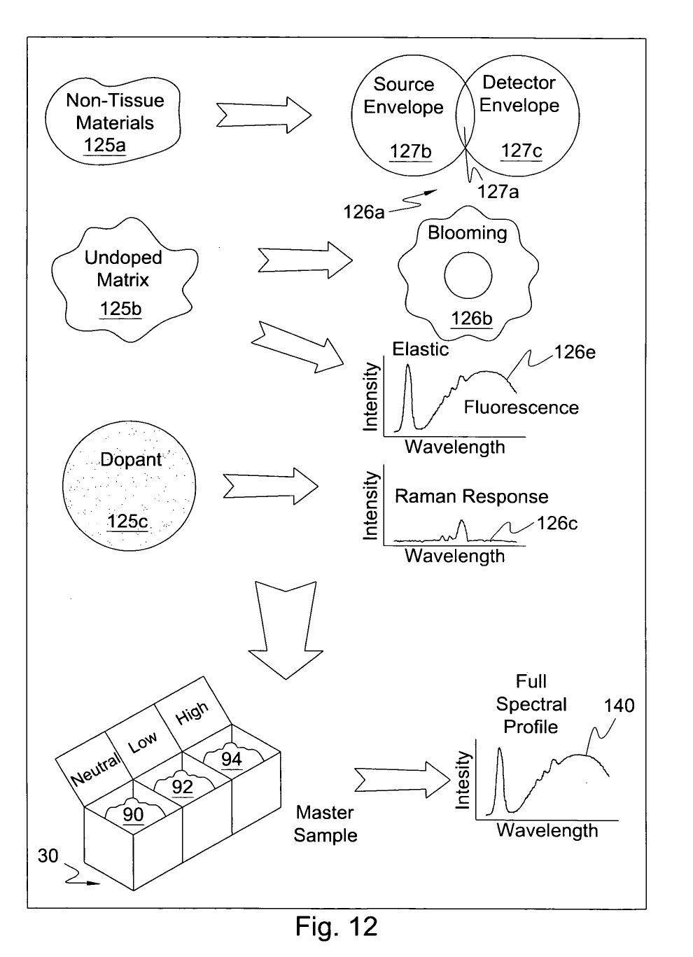

Referring to FIGS. 13-15, while continuing to refer generally to FIGS. 1-12, a chart illustrating a wavelength axis 132 as a domain, with an intensity axis 134 as a range, shows schematically the 473 nanometer wavelength 136 and the 510 nanometer wavelength 138. The 473 nanometer wavelength 136 is characteristic of a unique artifact in the curve 140 of radiant response, namely the elastic scattering peak 142, substantially centered thereon. Meanwhile, a large dome 144 representing the fluorescence response 144 of the curve 140 extends on either side of the characteristic 510 nanometer wavelength 138.

In general, elastic scattering as illustrated in the elastic curve 142 may be thought of as reflected light at the incoming frequency of a beam 101 projected from the window 14. Recall that frequency and wavelength are reciprocal and thus interchangeable, and both may be discussed as the domain over which data is taken. The fluorescence portion 144 of the curve 140 may be thought of as the re-radiation of light at a frequency different from that absorbed, typical of the surfaces of certain materials, including some rocks, and human skin.

Comparatively smaller preliminary peaks 146, 148 represent Raman scattering at wavelengths below the characteristic 510 nanometer wavelength 138. A substantial and recognizable peak 150 represents the Raman response surrounding the 510 nanometer wavelength 138.

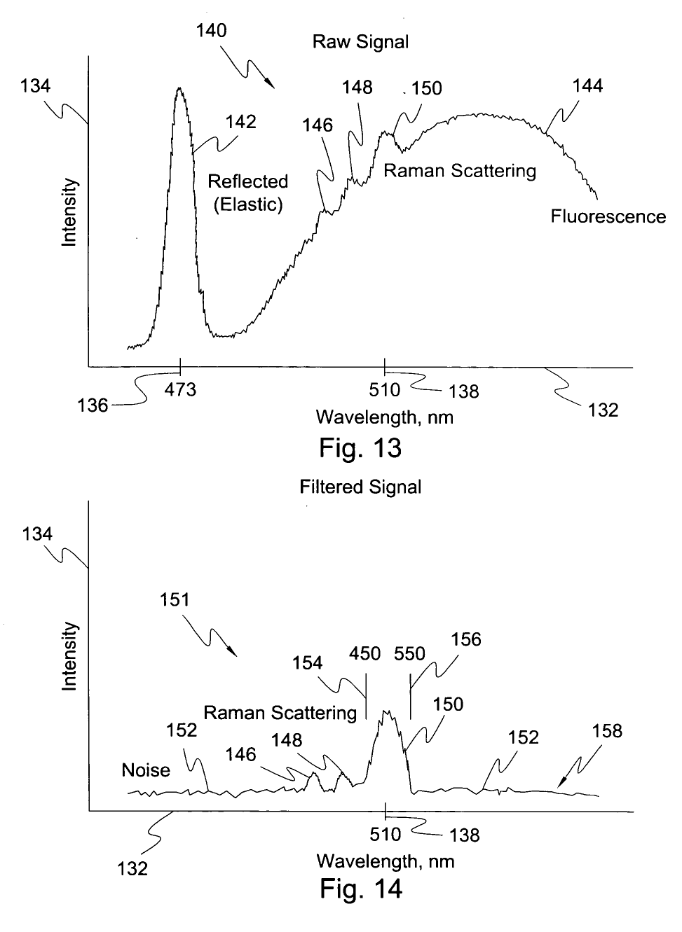

Referring to FIG. 14, a schematic of the Raman scattering portions 146, 148, 150 of the curve 140 may be subject to some degree of background noise 152. Nevertheless, by correction of the original radiant response curve 140 to remove the elastic scattering portion 142 and the fluorescence portion 144, the signal-to-noise ratio of the Raman scattering curve 146, 148, 150 is substantially improved. Typically, the region of interest extends around the 510 nanometer wavelength region from about 450 nanometers establishing a lower boundary 154 to about 550 nanometers establishing an upper boundary 156 of interest.

To obtain the curve 140 of FIG. 14, one may conduct a white field normalization. This may be accomplished by scaling the white scan curve 140 (based on scanning an undoped neutral sample 90) to the active scan curve 140 (based on scanning a doped or active material sample). The white scan may then be used to eliminate the effects of elastic scattering portion 142 and the fluorescent portion 144. The white scan curve 140 may be subtracted from or divided into the active scan curve. Subtraction may be problematic since small differences of large numbers are involved. Dividing the white scan into the active scan results in normalizing the common effects in the two curves 140, putting the peaks 146, 148, 150 in relief as shown in FIG. 14.

Although the overall curve 151 of FIG. 14 illustrates a nearly level or constant baseline curve 158, this is not necessarily so. That is, a baseline curve may actually have a slope that is non-zero. Nevertheless, in a schematic illustration, proportion may be necessarily exaggerated or minimized.

A white field normalization scan or a white scan may serve to adjust (e.g. calibrate) the control parameters of an apparatus 10 in order to assure that all such devices 10 provide the same output for a scan on a given calibration subject 30. Having the undoped matrix 125b of a neutral sample 90 or white scan sample 90 provides an ability to create a white scan in order to normalize the substantive data from active materials (e.g. doped, active, etc.). White scans may be used much as a dark scan is made using the dark cap 22. The white scan may be used to extract out the effects of optical artifacts of the optical system on the apparatus 10, as well as the elastic scattering 142 and fluorescent 144 responses (radiant responses) of a subject. This corresponds to the dark scan result used to extract our or normalize the artifacts (e.g. errors, anomalies, background, noise, etc.) due to electrical and electronic effects of the scanner 10.

Applicants have discovered that the dilatant compound serves as a neutral sample 90 (e.g. undoped matrix 125b) , doing for optical artifacts and background radiant responses a comparative neutralizing or normalizing function similar to that which a dark cap (e.g. dark scan) does for electrical artifacts. Removing these effects from the curve 140 can enhance the signal-to-noise ratio of the resulting Raman scattering curve 151. In this respect, a white scan may be considered as a “filtering” mechanism.

For example, the elastic response portion 142 and fluorescence portion 144 of the curve 140 represent part of the total radiant response of a subject (person, sample, etc.) to a light beam 101 from the window 14. The white scan sample 90 or neutral sample 90 that forms the undoped matrix 125b provides the two portions 142, 144 to be normalized out. Accordingly the curve 140 from a white scan may be scaled (sized, adjusted in range value in the chart) to match the curve 140 resulting from an active scan (using doped samples 92, 94, etc.) and divided into it.

The two curves 140 may be divided point by point (or by any other suitable means) into one another. The two curves could be subtracted, but small differences between comparatively large numbers may cause significant limitations on signal-to-noise ratios. White field normalization or flat field normalization typically relies on an initial scaling of the two curves to fit the same range, followed by division to provide a new normalized curve in which the relative orders of magnitude of points (range values at any location in the domain) are about equal. The division yields a result reasonably near unity. This typically enhances signal-to-noise ratios substantially, and highlights differences between active materials (e.g. samples 92, 94) and white samples 90.

Thus, a white field normalization or white scan using the neutral sample 90 is effective to provide a curve 126e to calculate out the effects of background, electronics, other wavelengths, intensities, optical aberrations, noise, and the like not already removed by the dark scan data. Together with dark scan reduction of electrical and electronic artifacts in the curve 140, scans of natural subjects can be relied upon.

Biological variations, perishable decay, and the like do not occur in the synthetic samples 90, 92, 94 of the master sample 30. Nevertheless, the dilatant compound has substantially the same fluorescence and reflectance as skin, notwithstanding the fact that the silicones themselves might have otherwise be optically clear.

The undoped matrix 125b that forms the neutral material 90 of the master sample 30 is actually effective to absorb solidus organic materials (e.g. foodstuffs, crystals of nutrients, etc.), and may also absorb liquid materials. In some embodiments, the dilatant compound forming the neutral sample 90 actually contains a small amount of water. Alcohol, acetone, or other solvents, such as carbon tetrachloride and the like, may be used in order to introduce materials into the dilatant compound.

The base material 90 or neutral sample 90 becomes a skin mimic, yet has substantially no “carotenoid mimic” absent proper dopant 125c. Applicants have found satisfactory a solid particle size that will pass 100 percent thereof through a number 200 mesh sieve. Substantially uniform particles blended uniformly throughout the matrix 125b (neutral material 90) seem to provide uniform results from the low value samples 92 and high value samples 94.

It is significant likewise that absent white field correction or “white scan” correction, excessive nonuniformity occurs in samples. Solid crystals were found to be somewhat unreliable, and provided no blooming effect 126b. Liquids were typically found to be absent sufficient uniformity or opacity to provide reasonable results. A hand may be used to calibrate several machines based on the uniformity of that hand. The ability to adjust multiple machines to that hand is limited by availability and uniformity. A single sample of a suspended material in a liquid has not typically provided reliability either. Using a dopant 125c along with an opaque material may provide a suitable liquid suspension.

Nevertheless, the viscoelastic material of the dilatant compound has shown proper opacity, radiant response, elasticity, adhesive qualities, and ability to suspend and distribute evenly a dopant 125c. It has shown an uncanny ability to mimic the reflectance portion 142 and fluorescence portion 144 of a radiant response 140 substantially equivalent to that of skin.

One caveat in scanning dilatant compound as a neutral sample 90 or as a doped sample 92, 94 relates to the Raman scattering peak 150 in the curve 140. The principal peak 150 appears to relate to a double carbon bond. The peak 148 appears to result from the Raman scattering from a single carbon bond. Likewise, the peak 146 appears to result from a single carbon bond attached to a methyl group. All peaks 146, 148, 150 are unlikely to be exactly matched at once. The value is also relative, regardless.

Calibrations can be done in any suitable scaling. For example, the base value of a curve 140 absent the elastic response 142 and fluorescent response 144 may be set at a value of zero. Meanwhile, a maximum value of a Raman scattering peak 150 may be set at a value of one. Similarly, a baseline could be set at zero, with a maximum value of one hundred. In one presently contemplated embodiment, a maximum value of the Raman scattering peak 150 may be set at a value or contribution value over sixty thousand, for example, sixty-seven thousand. Similarly, a low value may be set at the appropriate value in accordance with the maximum on a scale.

As a practical matter, the actual range of people based on a maximum value of intensity of about 67,000 was in part determined simply by an arbitrary scale approximating a photon count at a particular level of laser power. Actual values for scans of human skin on such a scale may range between a low of around 20,000, and a high on the order of 50,000. Of course, these vary from person-to-person. However, a range of zero to 67,000 is a suitable though arbitrary scale of intensity in one presently contemplated embodiment. It could as easily be scaled or mapped between any suitable interval, as known in the mathematical, signal processing, and other engineering arts.

For example, this range may be scaled from zero to one, minus one to one, from zero to ten, from one to ten, from one to a hundred, or the like. In other words, scale is always somewhat arbitrary. A value can virtually always be scaled

Dopant 125c may be added to a suitable undoped matrix 125b in order to provide suitable master samples 30 adequate to represent a range within reason as a standardized “synthetic tissue.” Having developed a standard for calibration, applicants have been able to standardize the outputs dependent on the radiant response of a subject to illumination by a beam 101 from the window 14. Until the advent of such calibration materials, structures, and methods, the number output by the apparatus 10 was simply an arbitrary number, having marginal interpretive value.

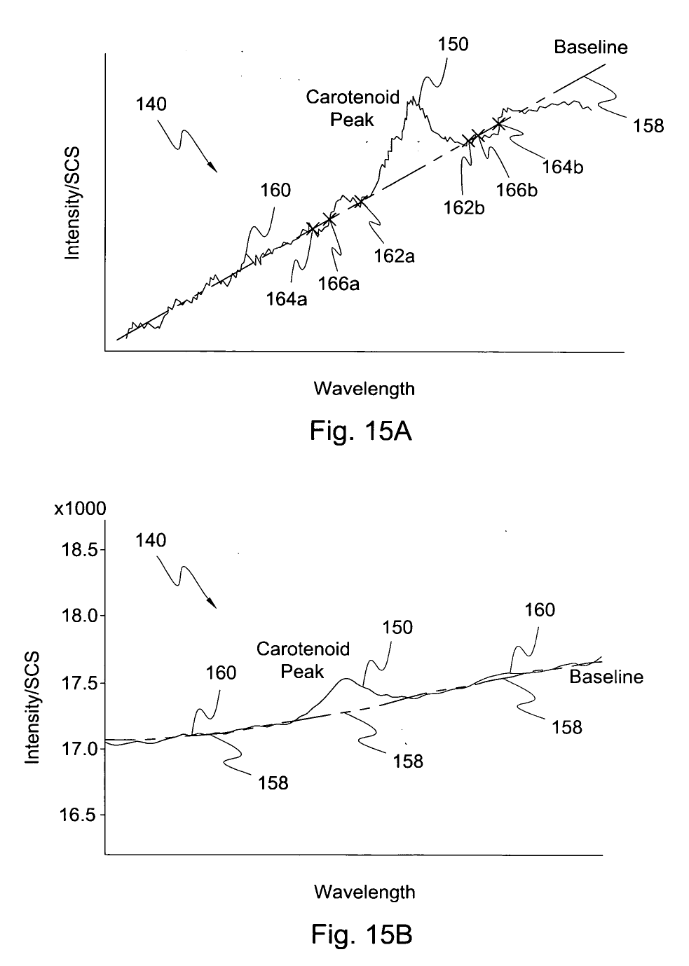

Referring to FIGS. 15A-15D, a curve 140 of radiant response, corrected for electronic and electrical artifacts, reflected or elastic light portions 142 and fluorescence 144, may be characterized by a data curve 160 remaining. The data curve 160 may be fit by suitable numerical methods with a baseline curve 158. The shape and order of both curves 140, 160 may be of a suitable order. A third order baseline curve 158 has provided a suitable fit. Higher and lower orders have been used successfully, but higher orders may produce anomalous peaks as mathematical artifacts. Lower orders may provide too gross a fit for comparison of the peak 150 against the underlying curve 160.

To form a baseline curve 158, the influence of the carotenoid peak 150 or the Raman scattering peak 150 of interest is best not included. Boundary points 162a, 162b may be selected to remove the peak 150 from consideration. That is, the boundary points 162a, 162b may bound the peak 150 in order that points therein not be included in the curve fit for the baseline 158. Similarly, extrema 164a, 164b may be selected. Intermediate points 166a, 166b are typically included at the sampling periodicity between a respective inner bound 162 and their respect outer bounds 164. Typically, approximately twenty points are included between the bounds 162, 164 on each side of the peak 150. A baseline curve is fitted through all the points 162, 164, 166. Thereafter, the intensity at the highest value of the peak 150 may be compared against that of the baseline 158 therebelow.

Referring to FIGS. 15B-15D, actual curves 160 have been corrected according to dark scans, white scans and appropriate normalization as discussed above to remove unwanted artifacts and effects in the data. The baseline curves 158 fitted, the curves 160 to which they are fitted, represent actual scans on a low value sample 92, an actual hand (in vivo subject), and a high value sample 94.

In calibration, a curve 160b may be a standard to which a machine is to be calibrated. Accordingly, a curve 160a may be the actual curve. By enforcing a map between the curve 150a and the curve 150b, or more particularly, by aligning the peaks of the curve 150a and 150b, the curves 160a, 160b may be substantially aligned. Moreover, the peak region 150 will be most accurately mapped.

Accordingly, one may think of adjusting the calibration as correcting the slope and intercept of the curve 160a to match the slope and intercept of the curve 160b. That is, assuming a linear curve fit, a rotation will result from correction of slope, and a translation will result from a correction of intercept. In the actual apparatus 10, parameters may be adjusted in order to adjust the coefficient and signal subtract or the “slope and intercept” for the baseline curve 158 underlying the peak region 150 of most interest.

Calibration accomplishes at least two purposes. Global consistency between machines (inter-machine consistency) is provided by standard reference materials. This takes a baseline 158 for a machine that can be relied upon in the future for field calibrations. The standardized settings for the apparatus 10 may then be set in order to achieve from a standardized master sample 30 the proper baseline curve 158 for any individual apparatus 10.

An apparatus 10 may have identified with it something on the order of sixty individual parameters identifiable and unique to that machine. Factory calibration accordingly sets parameters so that any two machines may read the same master sample 30 the same. Likewise, the other controlling parameters of the apparatus 10 may be adjusted in order that such an apparatus 10 may be used repeatably in the field. Accordingly, calibration in the factory of an apparatus 10 with its particular dark cap 22 and precision cap 24 assigned thereto will assure that the apparatus 10 may be field calibrated to its original factory specifications as needed.

The dark cap 22 provides a measure of the non-optical noise or background to be subtracted out. Similarly, the white scan material 90 or neutral sample 90 can be scanned to take out the fluorescence background, reflected light, and normalize the pixel-to-pixel variation in output from the detector (e.g. CCD, etc.). The low value sample 92 can be used to establish the low value (e.g. about 21,500 in one embodiment). The high value sample 94 may be used to establish the high level value (e.g. sixty-seven thousand).

In one embodiment of an apparatus and method in accordance with the invention, a computer, such as a laptop, PDA, or other processing connected to the apparatus 10, or embedded therewithin, may provide all of the calibration calculations, such that the hardware is not necessarily reset after the factory, or except at the factory. For example, once an apparatus 10 is calibrated at the factory, then the controls, illumination, and detection of radiant response from a subject can all be processed according to the calibration factors in software associated therewith. Accordingly, a CPU or processor embedded within, or attached externally to, the apparatus 10 may simply conduct all of the processing required in order to operate the apparatus 10. Thus, calibration may be more a matter of processing of parameters than actual operating parameters.

In one embodiment of an apparatus and method in accordance with the invention, many, many scans or illuminations and reading of radiant responses may occur with respect to a subject. For example, in one presently contemplated embodiment, one hundred seventy-five scans of approximately three hundred milliseconds duration (collection time for reading by a detector) may occur.

Within about a minute, something like one hundred seventy-five scans, images, pictures, etc. representing approximately three hundred milliseconds apiece of collection of data may occur. In general, Applicants have found effective the scanning of three such series. The average between those three processed series of scans, each representing one hundred seventy-five short scans of about three hundred milliseconds duration, have been found effective, repeatable, and reliable.

A scan time period substantially greater than three hundred milliseconds may saturate the sensors of the apparatus 10. A scan time of less than two hundred milliseconds by any substantial amount may tend to aggravate the signal-to-noise ratio of the resulting radiant response curve 140.

Applicants have found effective the use of certain curve smoothing algorithms. For example, a method sometimes referred to as the Savitsky-Golay method provides for smoothing of a curve, without destroying the peaks thereof. Accordingly, skilled operators can observe the peak region 150, and select the bounding points 162a, 162b. As a practical matter, considerable manual skill may be most effective. Nevertheless, numerical methods available may provide certain automated abilities. However, manual review has been found suitable in establishing the bounding points 162a, 162b, on either side of the principal peak 150. Thereafter, the baseline curve 158 may be fit.

The peak 150 may be fit with a polynomial. For example, third and fourth degree polynomials have been found suitable. Thus, the highest value pixel of the highest value reading in the curve portion 150 may then be established as a maximum, from which the baseline value corresponding thereto is subtracted.

An individual machine may then be adjusted individually by multiplying the maximum value of that peak 150 over the corresponding baseline value. For example, the factory precision caps 124 may be accommodated by a suitable adjustment factor in order to scale a reading received from the radiant response of the detector of the apparatus 10 to the cap 24 to match a standard. As an apparatus 10 is used over time, various conditions may occur hour-to-hour or location-to-location. Warming a machine up provides a certain amount of reduction in variability.

Field calibration represents a comparison of the ratio originally established for the reading of the cap 24, compared to the current day-to-day reading achieved for that same cap 24 on the apparatus 10 to which it is tethered.

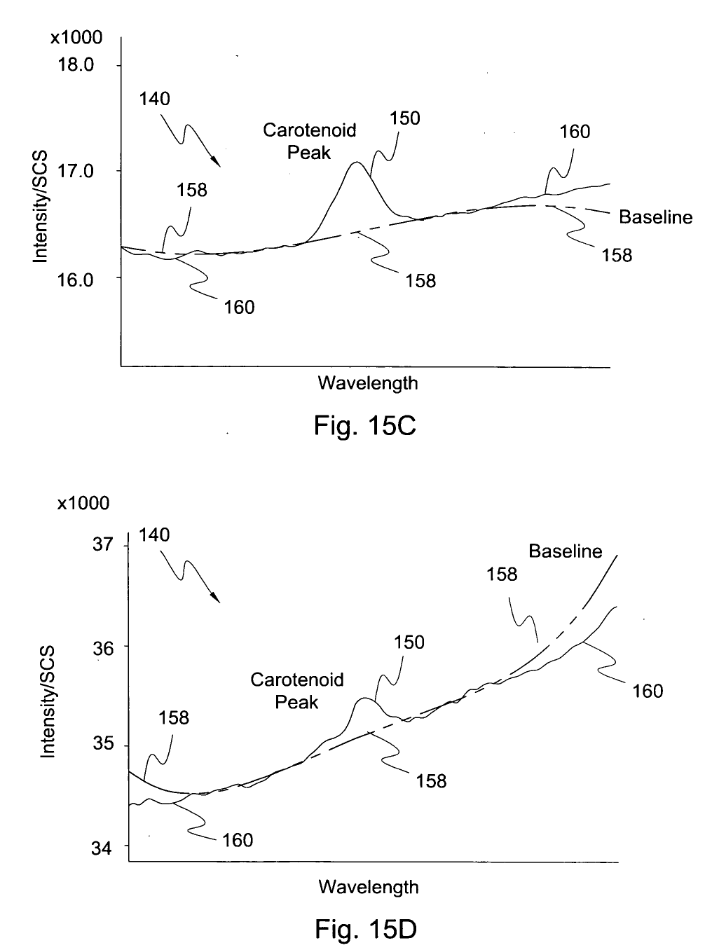

Referring to FIG. 16, an equation 168 representing a mapping of scale. A particular standard may be established against which other apparatus 10 may be calibrated, including being scaled. A laboratory unit or other device may be established as a standard. The numerical count (range, intensity, output, etc.) provided by the system or apparatus 10 is actually a reflection of intensity, a function of the number of photons impinging on a detector at a particular frequency and wavelength. Early devices bordering on laboratory curiosities were sufficiently sensitive to provide almost a count of photons. Thus a single count on a scale of zero to 67,000 was actually close to a count of photons impinging on a detector as a result of a scan.

The apparatus 10, need not be so sensitive as to accommodate and register arrival of every photon, so long as a measure of intensity is accurate and repeatable. Each apparatus 10 needs to read a given sample (e.g. master samples 30, live subject, etc.) and output a score or number identifying the same value for intensity of light detected. Thus, each apparatus 10 needs to be calibrated at the factory to match a standard. The advent of the synthetic master sample set 30 provides such a standard. This standard or master sample 30 is more reliable than data taken on biological samples, such as people or plant materials, since it is not subject to the vagaries of biological processes and degradation.

In FIG. 16, a skin carotenoid score SCS is a score or number corresponding to a reading achieved as an output of an apparatus 10. In calibration, this is the value output from reading the master sample 30. This is represented on the range (vertical) axis. The domain axis represents a value corresponding to the Raman scattering intensity obtained by a machine 10 under calibration to that same standard (e.g. a master sample 30).

A line may be defined by the peak heights 150 corresponding to scans conducted on the low and high samples 92, 94. The high sample 94 must read at the high value selected, (e.g. for example 67,000 in one embodiment) and the low sample 92 must read at the low value selected (e.g. at 21,500 in one embodiment). Other scales of numbers may be used, as discussed above, but these serve as one example.

Any resulting peak height 150 obtained on a machine 10 after calibration may be adjusted by the line of FIG. 16, mapping the output range of that calibrated machine to a set of standard values obtained from the same samples on a standardized test (e.g. apparatus). The map is made, resulting in a linear mapping equation during factory calibration. A coefficient (representing a slope M) and a signal subtract (corresponding to an intercept B) may be used to obtain the readout value (corresponding to dependent variable y) for any input readout value (independent variable x) from the calibrated scanner.

Thus any resultant peak height 150 obtained during a scan conducted by the calibrated machine 10 is scaled to the standard. This is accurate with only two points required for calibration, since Raman scattering is a linear effect. Accordingly, higher order terms are not required in order to map calibration scales of machines.

In practice, a dermal subject 172 is typically the palm of the hand of a person. Meanwhile, the content of molecular structures in serum 174 (e.g. bloodstream) in users can be correlated. The reading on a dermal subject 172 maps or correlates to the values determined by invasive evaluation of nutrient content (molecular structure of interest) within the serum 174 of the same subject.

Previously, laboratory developers of Raman scanning spectroscopy for carotenoid content could rely on comminuted tissues 176 from cadavers. Setting and fixing slides 177 is inherently subject to a lack of sample supply and repeatability for field calibration. Subject to irradiation, a factory sample has sufficient repeatability problems of its own. Irradiation sometimes affects the chemistry of carotenoids. Therefore, factory samples for evaluation of machines 10 may be problematic. Moreover, any hope for a repeatable, stable, sample from such a source is unthinkable.

Accordingly, applicants have used cuvettes filled with a liquid suspension 178 of synthetic materials, organic materials, and the like. The distance of the sample from the window 14 is problematic. Providing an opaque liquid suspension 178 helps solve that problem.

In fact, the dilatant compound matrix 180 (e.g. the neutral sample 90 of the master sample 30, or the undoped matrix 125b) provides the needed opacity, and is technically a liquid. The viscoelastic material flows under small force, albeit slowly.

The use of film 182, such as the films 110, 120 described above, and the layered system of materials 124 providing a response 123 or radiant response 123 to an incoming beam 101 have been found to be stable, predictable, and very useful. Nevertheless, the oriented nature (e.g. polarizing function) of these oligomeric films 182 makes them best suited for field calibrations of systems that have been matched thereto at the factory.

For example, because an apparatus 10 provides a beam 101 of nonoriented light, or of light having uncontrolled orientation, the apparatus 10 could be increased in complexity in order to assure a specific orientation of the light therefrom. However, as a practical matter, the peak 150 of interest in a response curve 140 calculated from the radiant response 123 to light beams 101 impinging on a sample of film 182 is unnecessary. So long as the particular sample 50 of film 182 is matched to a machine 10 and remains matched, the effects of polarization of the film sample 50 (e.g. 182) are repeatable and can be calibrated or accommodated into calibration.

One may consider why the distribution of a dilatant matrix 180 compounded as the master samples 30 might not be distributed to every operator of an apparatus 10. This is probably possible. Nevertheless, such a distribution constitutes a substantial amount of material, weight, and numerous control and protection issues. For example, manipulation of the master samples 30 may result in contamination, changed readings, and the like. By contrast, the synthetic films 182 represent substantially stable, protected, consistent calibration samples.

Other materials 184 may also be used. Nevertheless, opaque materials tend to be preferable, or at least materials that are sufficiently solid and responsive to fix distance effects. For example, as discussed hereinabove, samples 50 formed of film materials 182 can be used at different distances to represent different radiant responses, as if the distance were instead the molecular structure of interest at a different concentration.

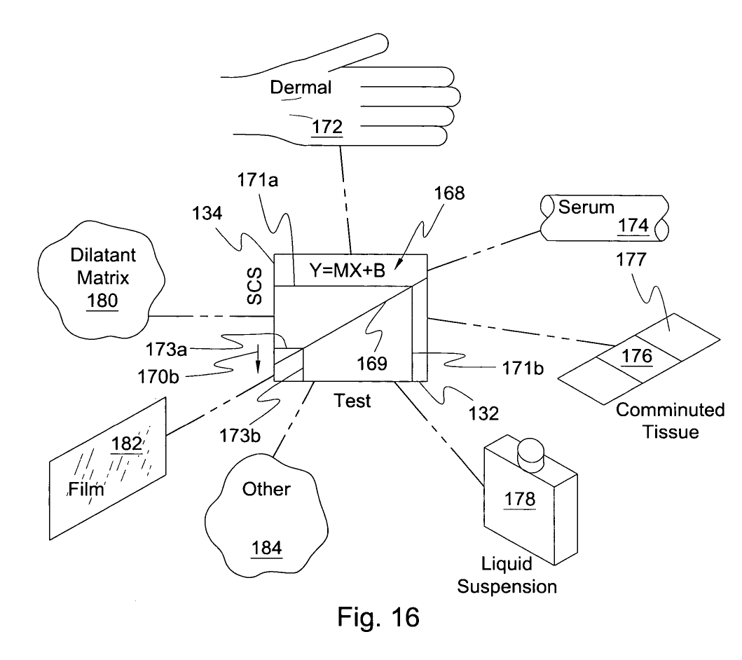

Referring to FIG. 17, a calibration process 188 may be thought of as a unit uniformity control process 190 and a condition uniformity control process 192. The unit uniformity control process 190 represents that machine-to-machine uniformity desired and achieved by a proper calibration in the factory. By contrast, the condition uniformity control process 192 represents the day-to-day or the session-to-session uniformity within a single machine.

As described hereinabove, a dark scan 194 may be followed by a background adjustment 195 of the controlling parameters associated with the apparatus 10, and the software processed in the CPU associated therewith, regardless of whether or not the CPU is embedded in or remote from the apparatus 10. Similarly, a white scan 196 results from an illumination of the neutral sample 90 by a beam 101, with collection of the radiant response 123 therefrom. Accordingly, the resulting data curve 140 may be used to make an adjustment 197 to the elastic and fluorescent portions of the data curve 140.

A factory sample scan 198 comprising either the low valued sample 92, medium 93, or the high valued sample 94 may be conducted, followed by the scan 199 of the other high value sample 94, medium values sample 93 or low value sample 92, respectively. Based on these data points, or more, if desired, a calibration adjustment 200 may be made. This calibration adjustment then accommodates the parameters and their readings affected thereby in the apparatus 10. The apparatus 10 and data processing are adjusted to provide an output therefrom matching a standard value of the curve 140, and the characteristic peak 150 off the baseline 158, when compared with other machines using the same master sample 30.

The condition uniformity calibration 192 may be done in the factory, with a precision cap 24 that will be tethered to the apparatus 10 for its operating life. The condition uniformity testing 192 may begin with a dark scan 202, or may rely on the original dark scan 194. Nevertheless, the condition uniformity calibration 192 in the field typically begins with a dark scan 202, in order to accommodate any conditional variation in the apparatus 10 or its environment during the particular time period of the scanning session for which condition uniformity calibration 192 is occurring. Following a dark scan 202, a background adjustment 203 is made to correct out from the data curve 140 the artifacts and other anomalies in the electrical and electronic operation of the apparatus 10.

Thereafter, a field sample scan 204 of a sample 50 embedded in the cap 24 or precision cap 24 is conducted, followed by a scan 205 of the alternate sample. That is, a high value and a low value sample 50 will be scanned 204, 205, in either order, as suitable. As a practical matter, all references herein to the precision cap 24 include the use of an alternative embodiment such as the spring-loaded cap 26, the double-ended cap 28, or the like. In the precision cap 24, or any of the other caps 26, 28, different values of samples 50 may be used on opposite ends or in alternate tries.

Nevertheless, one embodiment of the spring-loaded cap 26 was designed specifically to rely on distance for the variation in response 123 or radiant response 123 to the incoming beam 101. Likewise, either distance may be relied upon or concentration values of the samples may be relied upon to obtain the variations between high and low performance values of samples 50. Following at least two scans 204, 205, a calibration adjustment 206 may be made to adjust the value of the output numbers representing the characteristic peak 150 of interest.

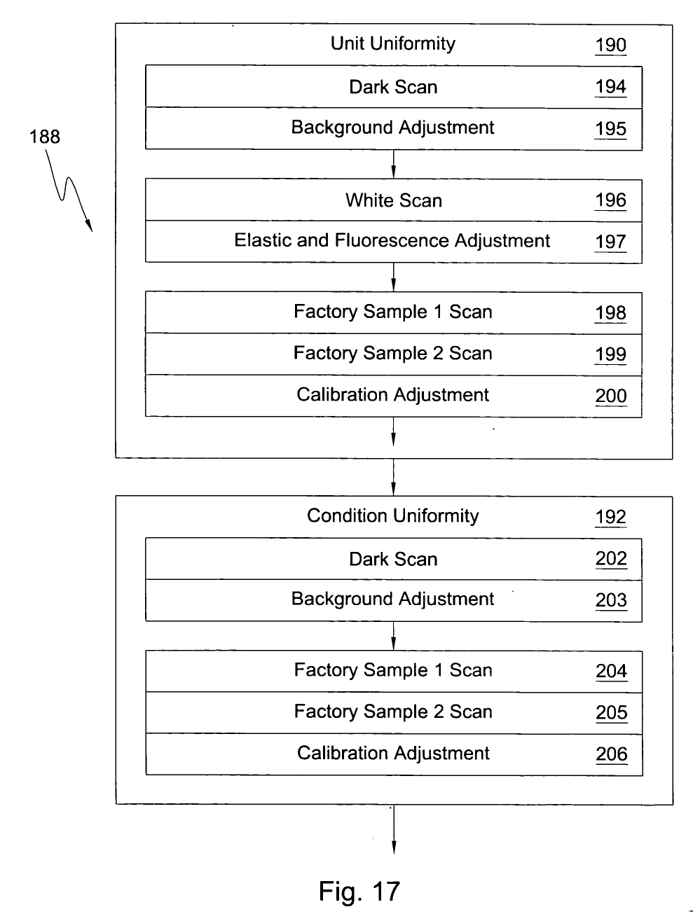

Referring to FIG. 18, a process 210 for creating master samples 30 may include selecting materials 212. This may include selection of a suitable material for a matrix 125b, as well as a suitable dopant 125c. By the same token, multiple matrices 125b, or multiple constituents for a single matrix 125b may be selected. Likewise, one or more dopants 125c may be selected for compounding and distribution or suspension in the matrix 125b.

After selection 212 of materials, including suitable testing, and other evaluations, preparation 214 of the matrix 125b may be done to order. This may be done by a supplier capable of delivering repeatable batches of the matrix material 125b.

Preparation 216 of dopants may include, for example, formulation 217a of a proper chemical or molecular structure of interest. Likewise, formation 217b of such a dopant 125c in a suitable format may be required. For example, in one presently contemplated embodiment, a K-type film of the oligomeric, polarizing-type may be ground, cut, or sanded to a fine powder. In one embodiment, a four hundred grit emery paper having a closed face to preclude contamination grinds particles that will substantially pass through a two hundred mesh chemical processing sieve.

Thus, formation of such particulate matter may include mechanical structuring of the particles, sizes, and the like. Ultimately, sizing 217c may be very important in order to provide uniformity. Particle sizes that are too large may provide erratic results. Similarly, particles that are too small may not be cost effective or as controllable.

Ultimately, distribution 218 of the dopant 125 in the matrix 125b results in a full set of master samples. That is, the neutral sample 90 comprises an undoped matrix 125 in one embodiment, but may instead involve a different matrix 125b with some backgrounding dopant of interest. Likewise, the low, medium, and high samples 92, 93, 94 will typically involve different concentrations of dopant 125 calculated and tested to provide a particularly suitable and broad range of values near the higher and lower ends of the expected results. For example, a low value registering twenty thousand on a scale of zero to sixty-seven thousand, and a high value composition 94 registering about sixty thousand on a scale of zero to sixty-seven thousand have been found suitable. On such a scale, human subjects have been scanned and found to typically lie between readings of twenty thousand and fifty thousand. Outliers may exist above and below this range, nevertheless.

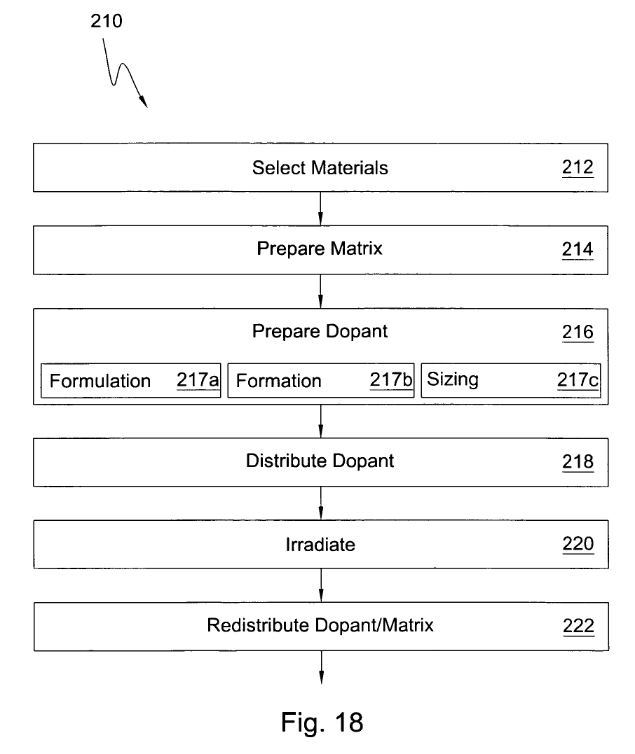

Referring to FIG. 19, an apparatus 10 and method in accordance with the invention may be implemented in a calibration process 224 in the field. The process 224 may initiate with activating 226 the scanner power to a position of “active” or “on.” Likewise, selecting 228 the process of scanning will typically be required. That is, somewhat independently from the scanner powering on 226, the activation 232 of a controller connected thereto may occur. Again, the processor (CPU) may be embedded within the apparatus 10, or may be a separate unit. Thus, the powering on 232 or powering up 232 of the controller presents a decision 234 after suitable delay.

After powering up 232, the controller may need to acclimatize for some period of time, such as about one-half hour. Thereafter, the scanner 10 is typically warmed up and prepared to operate. A user may then select between, for example, conducting a scan, uploading data curves 140 from previous scans, calling for support, reading or outputting reports, or shutting down the operation thereof. Upon evaluation of the options, the test 234 results in a choice of either selecting 228 a scan, or some other operation 236.

Upon selecting 228 to scan, an operator may then load 230 the software for controlling the apparatus 10. Several processes may occur including initiating a scanning session, warming up, calibration processes, retrieving information from previous scans or general information, conducting additional scans without beginning a new scanning session, outputting results, and the like. Accordingly, a user may navigate 238 operations to select a suitable operation.

In the case of conducting of scans, a dark scan 240 may occur first during the calibration process. A reference scan 242 followed by a second reference scan 244 will rely on the precision sample 24 (e.g. cap, spring-loaded cap, double-ended cap, or the like, etc.) in order to support calibration 246 of the specific apparatus 10 under the conditions of this particular scanning session. A quality control check 248 may be conducted on one or more actual subjects in order to verify that readings are operating within the expected ranges.

In one embodiment, control of the apparatus 10 may rely on entering a certificate number 250. The certificate number 250 supports the control of the use of the apparatus 10 in accordance with patents, licenses, and the like, in effect. Beginning either before or during the actual scan, inputting 252 the demographics associated with the subject may include tracking information that will be useful to the scanning operator, the subject, or both. For example, as data is collected anonymously from multiple subjects, additional assistance may be provided for characterizing relationships between intake, serum levels 174 and dermal levels 172 of the subject molecular structures (e.g. carotenoids, antioxidants, nutrients, minerals, amino acids, and other molecules of interest, etc.).

A hand of a subject is positioned 254 in front of the window 14, typically resting on the deck 16, or rest 16. The scanning 256 of the subject may occur as described hereinabove with hundreds of “scans” over a period of a few minutes in order to obtain a suitable, statistically significant sample. Processing 258 of the data then occurs in order to create the output curves 140 and to identify the value of the peak 150 of a baseline as discussed.

The test 260 determines whether scanning is complete for this session. If not, then entry 250 of another certificate number identifying a new subject permits continued operation. Otherwise, a test 262 determines whether or not a new session will be started. For example, a session may be shut off because the operator is going to change, the group of subjects is going to change, or calibration may be appropriate after some extended period of operation. If a new session is not to occur, then the apparatus may end 264 operation.

If a new session is to be conducted, then a test 266 may determine whether or not the time, number of scans, or other parameter for controlling use of the apparatus 10 has expired. If the test 266 determines that the time has not expired, then a new session may begin, with a dark scan 240 and other scans 242, 244 in order to complete calibration 246. On the other hand, if the test 266 results in a finding that the timeout or maximum number of scans or other parameter of control has expired, then a test 268 determines whether the currently scanned data will be uploaded to a server. If no upload is to occur, then the system will typically be disabled 270.

If, on the other hand, the data from the curves 140 is to be uploaded, then an upload process 272 occurs. Likewise, at the time of an upload 272, the overall process typically includes a download of authorizations for new scans, more time, or the like. Likewise, an optional step may include downloads 274 of upgrades to operational software. Thus, controller software, calibration schemes, and the like may be updated periodically for an individual operator.

Those of ordinary skill in the art will, of course, appreciate that various modifications to the detailed schematic diagram of FIGS. 1-19 may easily be made without departing from the essential characteristics of the invention, as described. Thus, the following description of FIGS. 1-19 is intended only by way of example, and simply illustrates certain presently preferred embodiments of a schematic diagram that is consistent with the invention as claimed herein.

In accordance with the foregoing needs, an apparatus and method are disclosed to calibrate a bio-photonic scanner to detect selected molecular structures of tissues, nondestructively, in vivo. The system may rely on a computer comprising a processor and memory connected to a scanner. The scanner includes an illuminator (e.g. light source, laser, etc.) to direct light nondestructively onto tissue in vivo. Light returns as fluorescence, reflective or elastic scattering, and Raman type scattering to a detector. The detector may be a charge coupled device or other mechanism to detect an intensity of a radiant response of the tissue to the light. A computer interface allows the scanner to communicate with the computer.

In certain embodiments, a calibrator contains a sample comprising a mimic material selected to mimic the radiant response of tissue. Determining a calibration parameter for the scanner may involve directing light from the illuminator onto the mimic material and detecting a first radiant response thereto. Inputs to the processor corresponding to a state of the light, the first radiant response to the light, and the calibration parameter enable calibration. Inputs are processed to repeatably detect a second radiant response of tissue in vivo as a result of exposure to light from the illuminator.

The method may include determining a calibration parameter, including selecting a curve corresponding to errors attributable to electrical artifacts and optical artifacts of the scanner to be corrected out of the radiant responses. The method may also include selecting a filtering parameter to filter out elastic scattering from radiant responses.

Selecting a curve corresponding to background fluorescence permits correction of this feature out of radiant responses. Points to define a curve corresponding to a radiant response, absent a Raman scattering response of interest therein may isolate a Raman scattering response of interest.

Typically, the light is coherent light from an illuminator such as a laser and the radiant response is an intensity corresponding to a selected molecular structure of the tissue, a constituent of interest, such as carotenoid materials, anti-oxidants, vitamins, minerals, amino acids, or the like. A Raman scattering response corresponding to carotenoids has been found effective. Moreover, calibration scans may be done using “mimic materials” of non-animal-tissue materials, structured to provide distinct readings different from one another. Different intensities can also be achieved for calibration by positioning one type of material at two different and distinct distances from the detector.

Samples found effective include various polymers, synthetic materials such as long chains, and oligomers used in polarizing filters. For example, tests have used K-type film and an HR type film manufactured by 3M company. Other samples include a pliable matrix containing a selected quantity of a dopant in different concentrations. The dopant may be a solid powder or a naturally occurring material, such as plant materials, vegetable derivatives, and the like. A powdered film sized to pass through about a no. 200 sieve has been found to form a good dopant.

A matrix of dilatant compound doped at two concentrations of dopant can receive naturally occurring material or a synthetic material. Effective synthetic materials seem to include a carbon-to-carbon bond corresponding to a similar bond in carotenoids.

Determining calibration parameters may include calculating correction curves to combine with data curves corresponding to the radiant responses of test (calibration) materials in order to isolate a “carotenoid” type of response therein. The correction curves may include data corresponding to at least one of elastically scattered light, fluorescence, and background artifacts of the scanner.

For calibration the machine is provided with a “dark cap” for collecting dark data in which substantially no light of interest returns to the detector, the dark data representing electrical artifacts of the scanner. Adjustments may be made according to the intensity of light from the illuminator, the response of the mimic material used in calibration, and correlation of the radiant responses of samples having different concentrations of dopants. The radiant responses to dopants are correlated between the sample and tissue in vivo.

In one embodiment, an operator may operate the scanner in a feedback control loop to detect in a subject an initial level of carotenoids in tissue. The subject may then ingest nutritional supplements according to some regimen over a subsequent period of time. Later testing with the scanner detects a subsequent level of carotenoids in tissue corresponding to the administration of the nutritional supplements.

Calibration of a scanner connected to a computer having a processor and memory may isolate a Raman response of carotenoids from elastic scattering, fluorescence, and electrical and optical artifacts of the scanner. A first synthetic material may be scanned to provide a “white scan” representing a portion of the radiant response of tissue attributable to optical artifacts of the scanner, reflected light, and re-radiated light at wavelengths not of interest (e.g. fluorescence). A suitable synthetic material is a viscoelastic material originally formulated by Dow Chemical and known as dilatant compound. In addition to serving as a neutral sample for conducting a “white scan” of background radiant effects, the dilatant compound may be doped at various concentrations.

In one embodiment of a system and method in accordance with the invention, a scanner of a bio-photonic type detects selected molecular structures of tissues, nondestructively, in vivo, from radiant responses of tissues to illumination by light from the scanner. The calibration system may include a dark sample returning a dark response corresponding to electrical artifacts of the scanner and comprising substantially no radiant response upon illumination thereof by the light. A white sample includes a first synthetic material returning a white response, upon illumination thereof by the light, substantially corresponding to a radiant response to the light of tissue, absent a characteristic Raman scattering response of interest.

A high valued sample may be formed of the first synthetic material treated with a dopant to return, upon illumination thereof by the light, a high response value corresponding substantially to a comparatively higher value of a radiant response of tissue to the light. A low valued sample may be formed from the first synthetic material treated with the dopant to return, upon illumination thereof by the light, a low response value corresponding substantially to a comparatively lower value of a radiant response of tissue to the light. The dark, white, high, medium, and low samples are each selected, formulated, and formed to provide parameters, which in mathematical combination calibrate the scanner, controlling computer, or both to provide a repeatable value of an output corresponding to molecular content in tissue in vivo in response to the light.

The basic synthetic material (e.g. matrix) is optically opaque, viscoelastic, silicone-based compound. It may include dimethyl siloxane, crystalline silica, a thickener, and polydimethyl siloxane as principle constituents. Decamethyl cyclopentasiloxane, glycerine, and titanium dioxide may be present in comparatively small amounts, and even a little water. The silicone chains are hydroxy-terminated polymers cross-linked by boric acid.

Dopants may be naturally occurring materials (e.g. carotenoids originating in plants, vegetables, foodstuffs, etc.) or a synthetic material. Synthetic materials having a molecular bonding structure corresponding to characteristic molecular bonding found in carotenoids seem to serve the purpose. One dopant is found to contain a chain of carbon bonds, including characteristic carbon-to-carbon double bonds. As a finely comminuted solid, the dopant suspends in the silicone-based matrix to mimic the Raman scattering and other radiant response properties of skin.

An apparatus for calibrating a scanner of a bio-photonic type may include hardware such as a dark scan structure, a factory calibrator of a standardized set of synthetic materials at different levels of doping, a field calibrator of a polarizing film, and a software executable in a computer-readable medium to receive and process data corresponding to scanning the dark scan structure, the factory calibrator, and the field calibrator. A computer programmed to run the executable calibrates the scanner and operates to control the scanner and output a value corresponding to the amount of the selected molecular structure based on data acquired during non-destructive scanning of tissue of a subject.

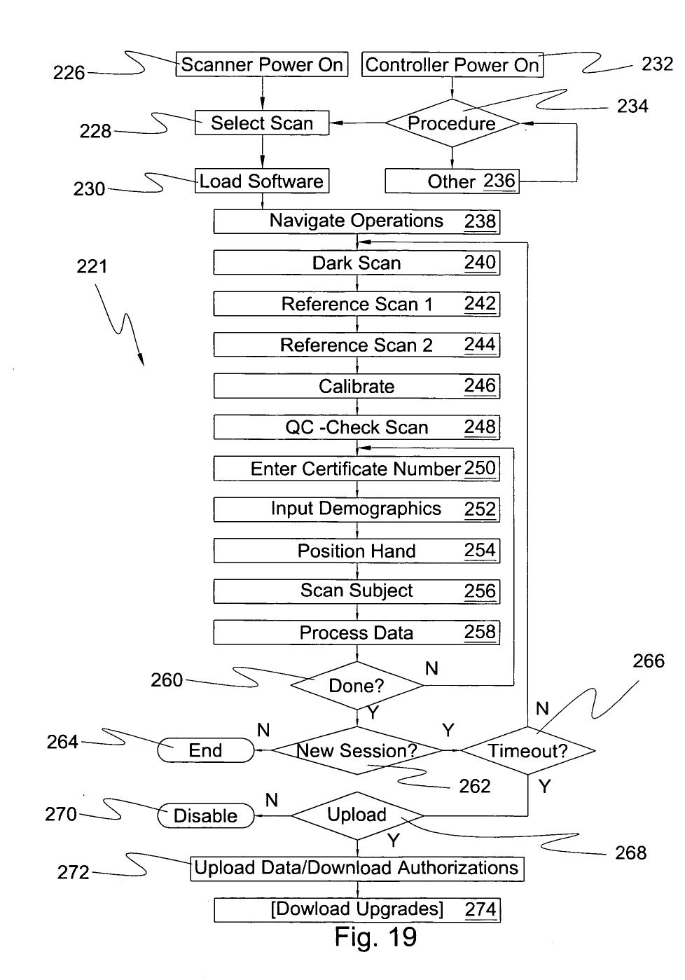

Referring to FIG. 20, a process 300 for establishing the calibration of a photonic scanner 10 may involve the use of tubes 302 containing a material for calibration. In one embodiment, each tube 302 may include a collar 304, driver 304, or the like to urge calibration material out of the tube 302. Each tube 302 may be provided with a lid 306 having an aperture 308 formed therein. The aperture 308 may size a sample 310 extruded therefrom upon rotation of the collar 304.

In one embodiment, the sample 310 may be formed 312, typically kneaded to an approximately round shape. For example, an operator may knead an approximately 1/4 inch ball of putty-like material for approximately 15 seconds. This forming process 312 may somewhat warm the sample 310 and provide additional mixing thereof Thus, forming 312 by kneading, rolling, or some combination thereof, may provide a ball 313 that may be inserted 314 into a cap 316. An operator should thoroughly wash and dry his or her hands prior to handling these synthetic calibration material standards and samples 310.

A cap 316 may be shaped and sized to engage and cover a window 14 or lens 14 of a barrel 13 of a photonic scanning device 10. In selected embodiments, a cap 316 may include an aperture 318 formed within a wall 320 or face 320. When inserted 314, the ball 313 may be positioned over the aperture 318 on the inside of the cap 316.

The shape and thickness of the face 320 into which the aperture 318 is formed may serve as a gauge for the ball 313 or glob 313 of test sample material 310. Typically, the ball 313 is on the order of between three and thirteen hundreds of a gram in mass. The face 320 may be sufficiently thin so the ball 313 of sample material 310 will extend inside the cap 316. Accordingly, a user may place a digit, such as a thumb or finger against the face 320 to covering the aperture 318 when placing the cap 316 over a lens 14 and barrel 13 of a photonic scanner system 10. The ball 313 may then deform between the user's digit, the edges of the aperture 318, and the window 14. Thus, a good complete contact is made against the window 14, without an individual touching the window 14, nor thinning out the ball 313 of material into a layer that is too thin to be properly opaque.

As a practical matter, a cap 316 in accordance with the present invention may have a rim 321 or collar 321 suitably formed to grip the barrel 13 associated with the window 14. An aperture 324 may be formed in an ear 322 extending from the collar 321. A lanyard 326 may connect to engage the aperture 324 in the ear 322. If desired, a lanyard 326 may include a loop 328 to preclude loss or to secure the cap 316 to the device 10.

In one embodiment of a calibration process, the dark cap may still be used. However, the two-sided calibration cap is replaced by a synthetic composition having a base composition of a silicone compound (also known and dilatant compound, the material commonly known as Silly Putty™). Various levels of dopants may be mixed with the base. Dopants may be naturally occurring compounds, but synthetics used in the film calibration system have been found to be suitable and stable if ground to powder and mixed into the base. There are several of these calibration standards. Each represents a different level of dopant including a neutral or undoped sample. This material may be distributed in tubes 302, each tube 302 containing enough material for a selected number of calibrations. This avoids “cap placement problems” in calibrating the scanner.

Once a ball 313 or glob 313 of sample material 310 has been properly positioned within a cap 316, an operator may apply 330 the cap 316 flush to the window 14 of the device 10. The sample material 310 may then be scanned 332 and the machine 10 calibrated accordingly. The sample material 310 may be discarded after a single use to prevent contamination. The machine 10 may be calibrated using a “white” scan, a low scan, and a high scan, as was done with the film cap system.

Following a scan 332 or series of scans 332, an output 334 may be provided produced by the photonic scanning apparatus 10 with its associated computational system. Typically, multiple scans 332 are run on multiple samples 310. As a practical matter, a blank or “white” material 90 having pigment but no doping provides a neutral sample. This material contains the dilatant compound with the standard pigmentation. Thus, the sample material 90 provides a white scan similar to the fluorescence of skin but with a total absence of the carotenoid effect on Raman scattering.

The low sample 92 may provide a sample size in the range including a typical lower limit on the Raman scattering effect in human skin. Similarly, a high sample or high-valued sample 94 may include a composition providing a scanned reading or value approximating that typically occurring at the upper limit of typical human skin response to Raman scattering. Similarly, one or more middle range samples 93 may provide intermediate value of scanning results, corresponding to a typical mid-range Raman scattering output expected from a scanning system 10.

Once sufficient data has been collected from the outputs 334 from scanning the multiple samples 90-94 multiple times, a decision 336 may be made. That is, if sufficient data has been collected, calibration 340 for the system 10 may be completed. Accordingly, the system 10 may be calibrated 340 according to the curve fitting, background elimination, normalization, and so forth. Nevertheless, until sufficient data is collected, a continued selection 338 of a next sample 310 from the materials 90, 92, 93, 94 available will continue iteratively. This selection 338 may continue iteratively on a single material 90, 92, 93, 94, as well as an iteration from material to material 90-94.

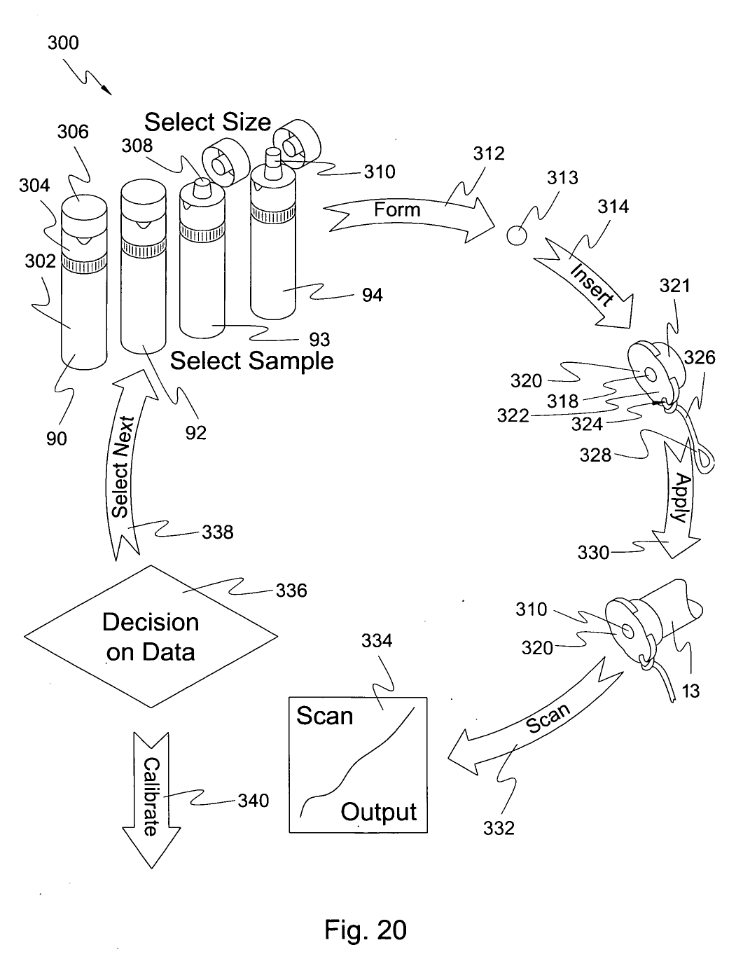

Referring to FIG. 21, the dopant materials may be formed independently from conventional processes previously relied upon. For example, in one embodiment, a process 350 for forming a dopant material may include dissolving 352 with a mixer 354 driven by a shaft 357 in a container 356. Power 358 or torque 358 to the shaft 357 may enhance the speed of dissolving polyvinyl alcohol (PVA) 360 in water 362. The addition of heat 359 to maintain about one hundred eighty degrees Fahrenheit within about fifteen to twenty degrees may also enhance the speed of dissolving PVA 360 in water 362. Additionally, crystal size may vary, but smaller crystal sizes for the PVA 360 enhance the speed of dissolving 352. An “eighty percent” PVA 360, granulated, of molecular weight of about 9,000 to 10,000, has proven effective.

Ultimately, the mixer 354, which may be any mixing device of any suitable type, will eventually provide a solution 364. In one embodiment, the PVA 360 may be from about ten percent to about fifty percent of the solution, and water 362 may be the remainder. A ratio of about eighty-five percent water to about fifteen percent PVA 360 has shown to be consistently effective and adequately fast.

The solution 364 may be passed to a tray 366 where evaporation 367 may occur, typically enhanced by the addition of heat 368. Drying over a period of hours with heat 368, or a period of days without heat, much of the water 362 evaporates 367 leaving a somewhat rigid sheet 370. Typically, the solution 364 may be poured out onto the tray 366 for a depth of from about one half centimeter to about one and one half centimeters. Typically, a depth of from about one quarter centimeter to about one centimeter has been shown effective. Accordingly, after evaporation 367, the sheet 370 may have a thickness of about one to two millimeters.

While still warm, or after warming, the sheet 370 may be cut by a shear 372 or cutter 372 of some other type to provide a particular size that may be handled by subsequent machinery or individuals. Each sheet 370 may be coated to form a coated sheet 374. In selected embodiments, the coating applied may be a spray or other application of hydrochloric acid (HCL) 376 to one or more sides of the sheet 370. A nozzle 378 or other tool 378, such as an applicator 378 available it the chemical arts may serve to apply the HCL 376. Application of reagent grade HCL 376 solution of about thirty-six to thirty-eight percent commercially available has been found effective across the surfaces of sheets 370 on the order of one to three millimeters in thickness. Any commercial HCL 376 should work.

The coated sheets 374 may then be placed in a furnace 380 substantially closed in by walls 382 and a door 384 or other access 384 in order to effectively apply heat 386 to the coated sheets 374. The heat 386 may typically be applied for about an hour to a few hours, typically no more than three or four. It has been found that approximately one hour of heat with a maintained temperature of approximately 160 C is effective to react the coded sheet 374 to make a dopant sheet 390. A range of plus or minus forty degrees Celsius will work but one hundred twenty slows reaction rates, while over two hundred degrees Celsius begins to carbonize the sheet 390. Typically, a dopant sheet 390 may be in large monolith, but may be in chunks, or granules. The dopant sheet 390 leaves the original clear appearance of the sheet 370 and the coated sheet 374 to become a deep and dark orange color. The orange color reflects the chemical condition of the dopant sheet 390.

A mill 392 driven by some set of drivers 394 and appropriate power 396 may then be used to grind the dopant sheet 390. The mill 392 may be one of any suitable type known in the chemical processing arts. For example, a cryogenic mill, a hammer mill, a grinder, rotary mill 392, or the like may all operate suitably. In one embodiment, a ball mill 392 has been found effective, using either spheres, cylinders, or the like. Similarly, the media used to operate in such a mill 392 has been suitably shaped as cylinders of various sizes, spheres of various sizes, and a mixture of both.

From the mill 392, following operation for a period of hours or days, a dopant powder 400 or dopant 400 in a powdered form results. A cryogenic grinder will provide results immediately. A ball mill may take from one day to five days. Typically, by selective choice of media, one day to two days of operation of a ball mill has been found effective to provide a comparatively high percentage of suitable dopant 400. That is, the size of the mean diameter typically becomes smaller for the particulates in the dopant 400 with more time in milling. Yields on the order of thirty percent to about seventy percent mass of particles of number three hundred twenty-five sieve size have resulted. Typically with at least some steel balls as media in a mill 392, yields of approximately sixty percent have been found consistently. Milling time may vary from about one to four days and is typically two days, if steel spheres are used, to get a sixty percent yield of number three hundred twenty-five sieved particles.

Following grinding or milling of the dopant 390 to form the dopant powder 400, sieves 402 may sort the dopant 400 by size (mean size of particles). A series of sieves 402 may be used, where each sieve 402a-402e includes a screen 404 having a particular clearance size or mesh size. Typically, with dopant powders 400 made as described hereinabove, a screen mesh size over three hundred has been found effective. Moreover, a screen mesh size of three hundred twenty-five is used regularly. Using a mesh size corresponding to any number high than three hundred has not been shown to provide any improvement in results. Nevertheless, mesh sizes on the order of a number two hundred have been used successfully.

A number two hundred seventy sieve and larger sizes do show more variation in the quality of Raman scattering from the dopant 400.

Each of the sieves 402 is formed of a wall 406 or rim 406 maintaining the screen 404 therein as a substantially planar material. The sieves 402 may be stacked in a stack 408 and placed in a vibration device as known in the art in order to enhance the speed at which the dopant 400 passes through each of the screens 404.

Ultimately, a sized dopant particulate 410 may result from the smallest sieve 402e used. Typically, a sieve having a number 300 has been found consistently suitable, with no substantial improvement noticeable by any smaller sizes. Nevertheless, in some commercial materials, a sieve mesh size as large as 200 has been found suitable. Nevertheless, the 200 mesh has been most suitable with materials that include the dopant material on a substrate, and thus may result in a suitable particle, of a larger size, whereas the dopant 400 is used in a more pure form, and thus may be comminuted to a smaller size to obtain the same concentration. Oversized dopant 411 may be recycled through the mill 392. In practice, no degradation has been observed in the oversized dopant powder 411 or particulates 411 by continued milling in the mill 392.

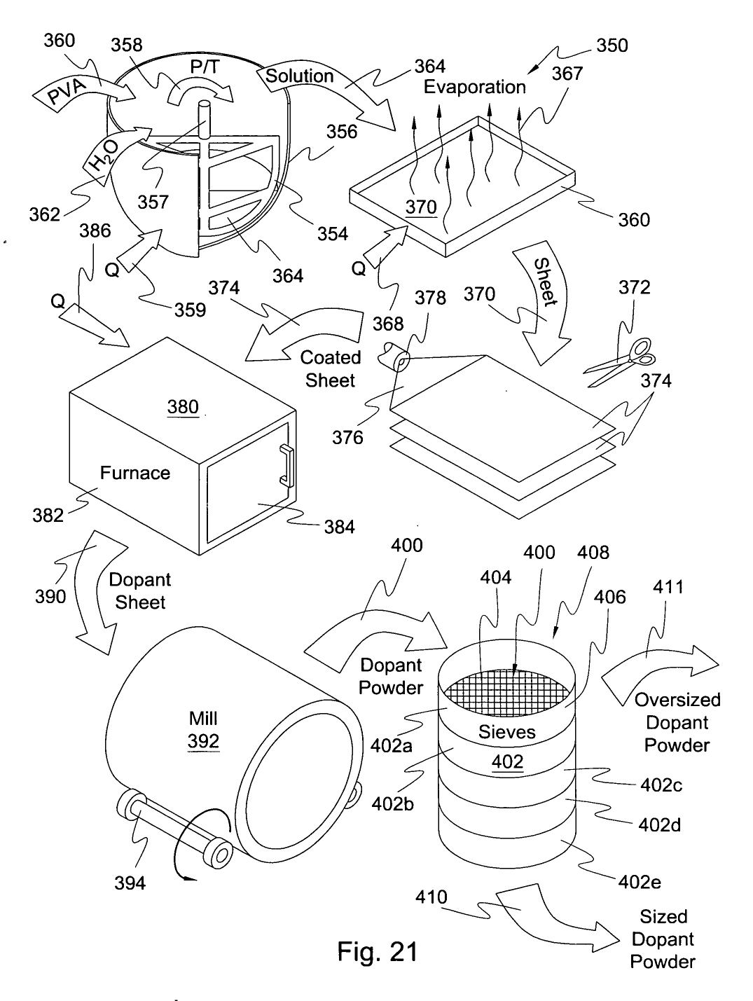

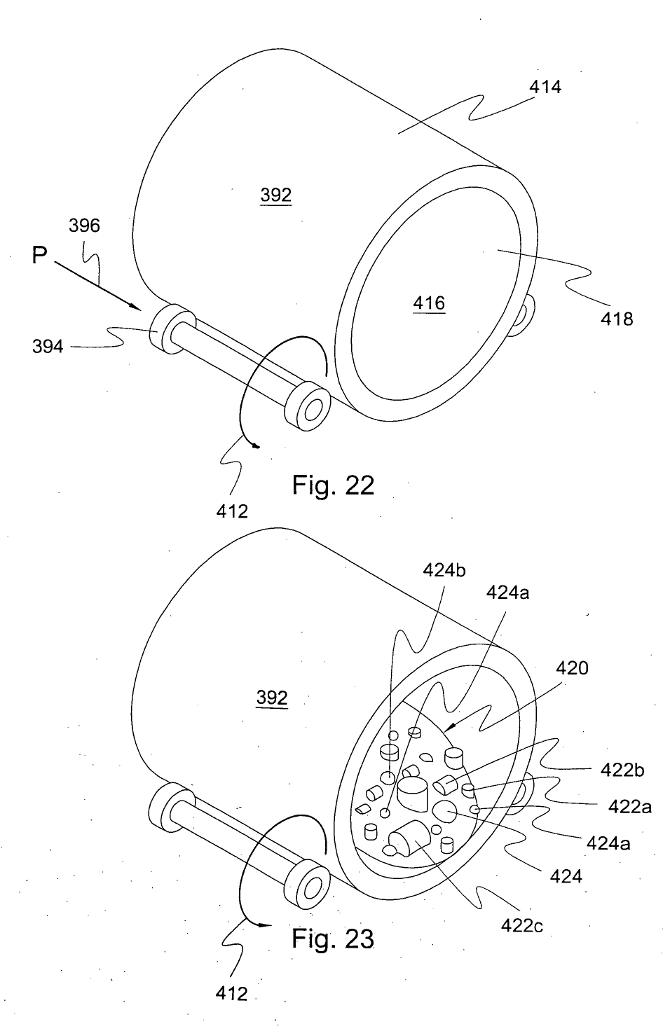

Referring to FIGS. 22-23, in one embodiment of a ball mill 392, a torque 412 may be applied to a driver 394 or set of rollers 394 driving a drum 414 or container 414. The drum 414 may have an access 416, such as a door 416 or other closure 416. Upon opening the door 416, an opening 418 may provide for feeding and removal of material and media. Typically, the media 420 may include various sizes and shapes of heavy and hardened materials. That is, ceramic cylinders, ceramic balls, steel balls, steel cylinders, and the like may serve a media 420.

Cylinders 422 have been found effective in multiple sizes, such as approximately one centimeter diameter by approximately one centimeter or to one and a half centimeters in length, as well as an intermediate size of approximately double those dimensions, and a larger size of approximately triple the dimensions of the smallest units. For example, diameters on the order of one centimeter, two centimeters, and three centimeters seem to serve, while lengths from approximately equal thereto up to about fifty percent greater have been found suitable. Similarly, steel balls of those dimensions appear to serve adequately. A mixture thereof also works well. Ceramic or steel cylinders, spheres, or a mixture thereof may serve well.

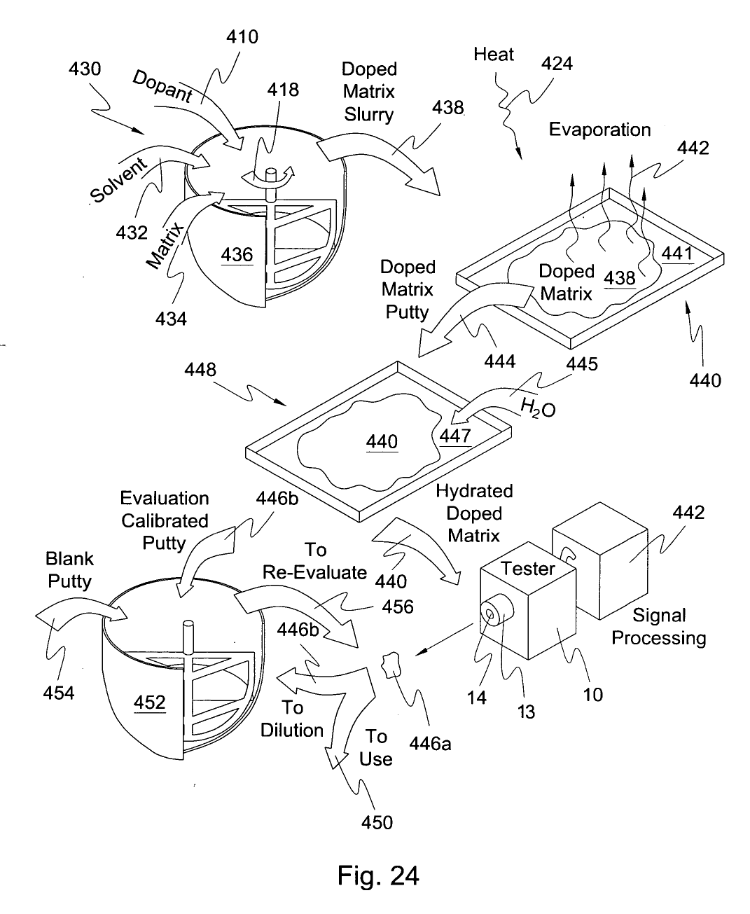

Referring to FIG. 24, a process 430 suitable for formulating the master samples 30, such as the low valued sample 92, medium valued sample 93, and high valued sample 94 may begin by adding a solvent 432 to a matrix 434 of dilatant compound 434. The matrix 434 may benefit from approximately an equal mass or volume of a solvent 432 such as ethanol. Sufficient solvent 432 may be added to the matrix 434 in a mixer 436 to provide a slurry 438 easily poured. Thus, a consistency similar to that of cake batter or other slurries has been found suitable. Since the solvent 432 will eventually be evaporated, the amount of solvent 432 does not need to be controlling, so much as the ability to handle the slurry 438. Blending may be done in a matter of minutes, typically about ten minutes, depending on equipment and batch size.

The matrix 434 in one embodiment has been mixed with a dopant 410 in order to provide a completely doped slurry 438. In certain embodiments, the dopant 410 may be added without the solvent 432. Nevertheless, clumping of the dopant particles 410 has been observed. Observable static electricity, particularly in the presence of the matrix 434, leads Applicants to believe that the dielectric nature of both the dopant 410 and the matrix 434 may be assuaged or ameliorated by the addition of solvent 432. The solvent 432 may act as an electrical conductor or may simply act as a material to exert surface tension over the particulate dopant 410, while also rendering the slurry 438 more easily handled.

The slurry 438 may be delivered into a chamber 440 containing a tray 441. That is, a chamber 440 suitable for drawing off or conducting off the solvent 432 in an evaporation process 442 results in a suitable putty 444. If desired, the doped putty 444 may have added to it a certain amount of water 445 in order to form a hydrated putty 446. Typically, a mere ten milliliters to about thirty milliliters per kilogram of the doped putty 444 has been found effective to form a more easily handled and shaped hydrated putty 446. Typically, ten milliliters of water in half a kilogram of the doped putty 444 has been found to provide a suitable hydrated putty 446 upon proper kneading 448 in some suitable container 447, mixer 447, or the like. Kneading 448 may be done by a machine, or by hand, depending on the quantities desired.

The hydrated putty 446, constituting a doped matrix of dilatant compound may be scanned in small samples 446a by irradiation through a lens 14 of a barrel 13. Processing by a suitable computer system described hereinabove may provide an evaluation. Thus, a determination may be made as to whether the sample 446a is suitable to use 450. That is, sample 446a may have the proper doping to become one of the master samples 30 (90, 92, 93, 94).

Nevertheless, it has been found that above approximately sixty grams of dopant 410 per kilogram of matrix 434, the Raman scattering is excessively bright. Accordingly, if the Raman scattering from the proper irradiance by the apparatus 10 is higher than desired, then the material 446a may be sent as dilution material 446b. That is, the batch of hydrated, doped-matrix putty 446 may be sent to a mixer 452. The mixer 452 may be of any suitable type, from manual kneading to commercial, powered mixers 452. In the mixer 452 or an associated process, blank putty 454 corresponding to the matrix 434, or a hydrated amount of the matrix material 434 may be added to the evaluated putty 446b to form a new diluted putty 456 later re-submitted for reevaluation 456.

Applicants have found that once a particular concentration of dopant 410 has been properly kneaded into or mixed into a hydrated putty 446, it can be “cut” (diluted) by any suitable amount of blank putty 454 containing no dopant, in order to obtain a precise percentage of dopant 410. Accordingly, the response from each sample 446a can be engineered to obtain a comparatively precise Raman response.

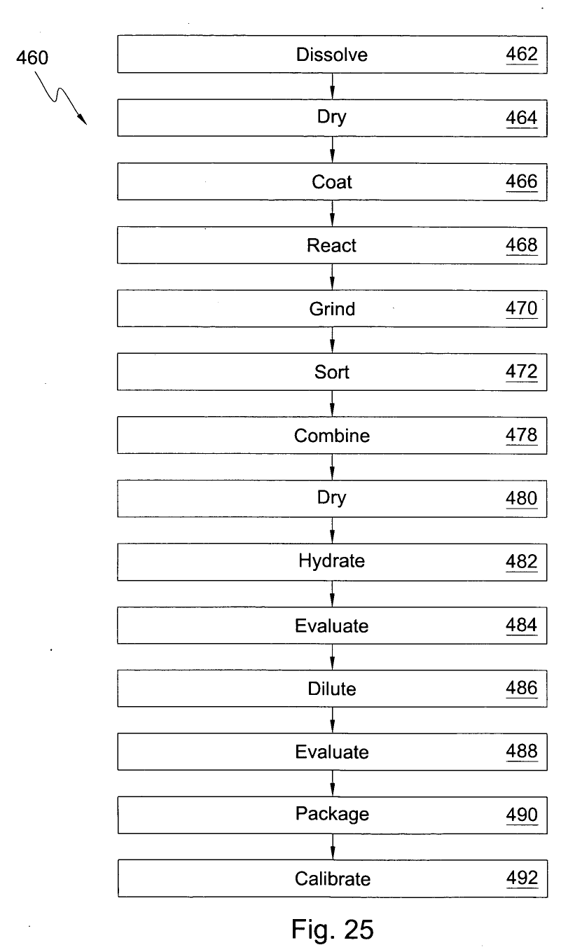

Referring to FIG. 25, a process 460 for preparation of a suitable putty for use 450 may involve dissolving 462 polyvinyl alcohol with water in a ratio of about one part alcohol to five parts water. This may be done by weight or volume, since the specific gravity of polyvinyl alcohol is only slightly above that of water.

Next, the solution resulting from dissolving 462 may be dried 464 by the application of ventilation, air, heat, or a combination thereof in order to provide a film on the order to one to three millimeters in thickness, in certain embodiments. Coating 466 these layers of sheet PVA 370 with hydrochloric acid provides the materials for a suitable reaction 468. The hydrochloric acid has been found to be a standard reagent grade. One typical embodiment involves from about thirty-five percent to about forty percent hydrochloric acid solution at a reagent quality. The reaction 468 may be enhanced by the addition of heat, and maintenance at a temperature of about 160 C. A variation in temperature of about forty degrees higher or lower may degrade the quality of the reaction. Thus, below 120 C tends to promote less vigorous reaction, whereas temperatures above 200 C appear to tend to degrade and carbonize the resulting material.

After the reaction 468, the resulting dopant sheets 390 may be ground 470 by any suitable process. Many suitable mills exist, and Applicants have found a ball mill to be adequate, although not particularly fast. Cryogenic grinding systems are used in the chemical processing industry, and may also perform the grinding function.

Following grinding 470, sorting 472 by a system of sieves 402 provides a sized dopant powder 410, as well as an oversized dopant powder 411. Oversized powder may be recycled through the mill 392, while the properly sized dopant powder 410 may be taken for use.

The process 460 may continue with a combination 478 or combining 478 the dopant 410 along with a matrix 438 of dilatant compound or other matrix 434 and a suitable solvent 432. Following a complete mixing 478, the mixture may be dried 480 by application of modest temperatures, and ventilation.

The resulting doped putty 444 may be hydrated 482 for easier handling and packaging. In order to establish a particular and specific amount of dopant 410 per gram or kilogram of the hydrated putty 446, a dilution 486 with blank putty 454 may introduce a proper amount of undoped putty 454. Following a repeat of the evaluation 484 and dilution 486 until an evaluation 488 results in a product suitable for use 450, results in a system 30 or master set 30 of samples ranging from a neutral 90 to a low 92, one or more medium-valued samples 93, and a maximum or high-valued sample 94. Packaging 490 may include placement of the suitably hydrated matrix material 456 ultimately resulting into a system of tubes 302 for easy extrusion and use of small samples 310 on the order of 0.1 to 0.01 grams each. Typically, a quantity from about 0.03 to about 0.1 grams has been found suitable, as has any larger amount. Thereafter, calibration may occur using the master samples 30.

The present invention may be embodied in other specific forms without departing from its essential characteristics. The described embodiments are to be considered in all respects only as illustrative, and not restrictive. The scope of the invention is, therefore, indicated by the appended claims, rather than by the foregoing description. All changes within the meaning and range of equivalency of the claims are to be embraced within their scope.

1. A system comprising:
a matrix comprising a first synthetic material formulated to return a fluorescence corresponding to living tissues upon illumination thereof by a source of light;
a dopant formulated to provide a characteristic Raman scattering response corresponding to that of a selected molecular structure in living tissues upon illumination thereof by a source of light; and
the matrix and dopant selectively mixed to provide samples having respective radiant responses effective to mimic radiant responses corresponding to those of living tissues over a corresponding range of amounts of the selected molecular structure in living tissues.

2. The system of claim 1, wherein the first synthetic material is optically opaque and comprises a pigment providing fluorescence substantially corresponding of tissue.

3. The system of claim 2, wherein the first synthetic material is a viscoelastic material.

4. The system of claim 3, wherein the first synthetic material comprises at least one of silicone, dimethyl siloxane, decamethyl cyclopentasiloxane, polydimethyl siloxane, titanium dioxide, and quartz crystalline silica.

5. The system of claim 4, wherein the first synthetic material comprises at least one of a thickener, glycerine, and water.

6. The system of claim 1, wherein the first synthetic material comprises at least one of a hydroxy-terminated polymer, boric acid, dilatant compound, and a compound of silicone oil and boric acid.

7. The system of claim 1, wherein the dopant comprises a naturally occurring material.

8. The system of claim 7, wherein the dopant comprises a carotenoid originating in a plant material.

9. The system of claim 8, wherein the dopant comprises a carotenoid originating in a foodstuff.

10. The system of claim 1, wherein the dopant comprises a material having a molecular bonding structure corresponding to a characteristic molecular bonding found in carotenoids.

11. A method of making a calibration standard to mimic a compound of interest detectable in live tissue by a scanner operated to provide photonic illumination of live tissue and detection of a response thereto, the method comprising:
providing a polyvinyl alcohol having a surface;
treating the surface with a reactant effective to form a dopant to mimic the compound when illuminated by the scanner;
combining the dopant with a carrier selected to provide a fluorescence corresponding to that of tissue when illuminated by the scanner to form the calibration standard; and
scanning the calibration standard to provide a value corresponding to Raman scattering by the dopant in the calibration standard in response to illumination by the scanner.

12. The method of claim 11, wherein providing a polyvinyl alcohol further comprises:
dissolving a polyvinyl alcohol in a first solvent to form a solution;
containing the solution on a substantially planar support to provide a pool of the solution having comparatively small aspect ratios of thickness with respect to length and width, respectively ;and
drying the solution to form a sheet of polyvinyl alcohol as a solid having surfaces.

13. The method of claim 12, further comprising elevating an ambient temperature of the solid to accelerate the reaction of the reactant and the solid.

14. The method of claim 11, wherein the compound is detectable non-invasively and non-destructively in vivo.

15. The method of claim 11, wherein the bio-photonic scanner is configured to render the compound detectable non-invasively and non-destructively.

16. The method of claim 11, wherein the first solvent is water.

17. The method of claim 11, wherein the carrier further comprises a pigment effective to reflect and absorb a selected range of light substantially as would tissue.

18. The method of claim 17, wherein the range of light is within the portion of the electromagnetic spectrum visible to humans.

19. The method of claim 11, wherein combining further comprises mixing the dopant and a second solvent with the carrier and drying the mixture to remove the second solvent.

20. The method of claim 11, wherein the reactant is hydrochloric acid.

21. The method of claim 11, further comprising diluting the calibration standard with additional quantities of the carrier to provide an array of calibration standards, each having one of a range of values corresponding to Raman scattering in accordance with the amount of dopant therein.

22. The method of claim 11, further comprising comminuting the dopant.

23. The method of claim 11, further comprising providing a container to hold the calibration standard, providing an extraction structure therein to extrude the calibration standard from the container, providing a measureable quantity thereof.

24. The method of claim 11, wherein the compound of interest is an antioxidant.

25. The method of claim 24, wherein the antioxidant is a carotenoid.
